(19) United States
(12) Patent Application Publication
(10) Pub. No.: US 2018/0012623 A1
(43) Pub. Date: Jan. 11, 2018

(54) Ionic Liquid, Lubricant, and Magnetic Recording Medium

(71) Applicant: Dexerials Corporation, Tokyo (JP)

(72) Inventors: Hirofumi Kondo, Tokyo (JP); Kouki Hatsuda, Tokyo (JP); Nobuo Tano, Tokyo (JP); Pankaj Baghel, Tokyo (JP)

(21) Appl. No.: 15/633,062

(22) Filed: Jun. 26, 2017

(30) Foreign Application Priority Data
Jul. 5, 2016 (JP) 2016-133680

(51) Int. Cl. G11B 5/725; C07D 233/74; C07C 309/06

(52) U.S. Cl. CPC G11B 5/725; C07C 309/06; C07D 233/74

(57) ABSTRACT

A lubricant including an ionic liquid including a conjugate base and a conjugate acid, wherein the conjugate acid includes a straight-chain hydrocarbon group having 6 or greater carbon atoms and a straight chain hydrocarbon group s having 6 or greater but 14 or less carbon atoms, and wherein a pKa of an acid that is a source of the conjugate base in acetonitrile is 10 or less.

### BACKGROUND OF THE INVENTION

### Field of the Invention

The present invention relates to an ionic liquid, a lubricant containing the ionic liquid, and a magnetic recording medium using the lubricant.

### Description of the Related Art

Conventionally, in a thin film magnetic recording medium, a lubricant is applied onto a surface of a magnetic layer for the purpose of reducing frictions between a magnetic head and the surface of the magnetic recording medium, or reducing abrasion. In order to avoid adhesion, such as sticktion, an actual film thickness of the lubricant is of a molecular order. Accordingly, it is not exaggeration to say that the most important thing for a thin film magnetic recording medium is to select a lubricant having excellent abrasion resistance in any environment.

During a life of a magnetic recording medium, it is important that a lubricant is present on a surface of the medium without causing desorption, spin-off, and chemical deteriorations. Making the lubricant present on a surface of a medium is more difficult, as the surface of the thin film magnetic recording medium is smoother. This is because the thin film magnetic recording medium does not have an ability of replenishing a lubricant as with a coating-type magnetic recording medium.

In the case where an adhesion force between a lubricant and a protective film disposed at a surface of a magnetic layer is weak, moreover, a film thickness of the lubricant is reduced during heating or sliding hence accelerating abrasion. Therefore, a large amount of the lubricant is required. The large amount of the lubricant is the mobile lubricant, and therefore a function of replenishing the lost lubricant can be provided. However, an excessive amount of the lubricant makes the film thickness of the lubricant larger than the surface roughness. Therefore, a problem associated with adhesion arises, and in a crucial case, sticktion arises to cause driving failures.

As illustrated in FIG. 1, although an increase rate of an areal density of a hard disk drive of a product have been reduced in the last few years, the increase rate has reached an anural rate of 25%, and has nearly reached 4 Tb/in², which is one of targets, in Advances in Tribology Volume 2013, Article ID 521086. As illustrated in FIG. 2, it has been understood that a distance between head disk interfaces relative to an increase in the recording density reduces, but there is a need to always improve reliability corresponding to the reduction in the distance, which can be described, for example, in the following literatures.
(<1>C. M. Mate, Q. Dai, R. N. Payne, B. E. Knigge, and P. Baumgart, “Will the numbers add up for sub-7-nm magnetic spacings? Future metrology issues for disk drive lubricants, overcoats, and topographies,” IEEE Transactions on Magnetics, vol. 41, no. 2, pp. 626-631, 2005.,
<2>B. Marchon and T. Olson, “Magnetic spacing trends: from LMR to PMR and beyond,” IEEE Transactions on Magnetics, vol. 45, no. 10, pp. 3608-3611, 2009., and
<3>J. Gui, “Tribology challenges for head-disk interface toward 1 Tb/in²,” IEEE Transactions on Magnetics, vol. 39, no. 2, pp. 716-721, 2003.)

A current recording density is about 1 Tb/in², spacing is about 6 nm, and a thickness of a lubricant is 0.8 nm. The thickness of the lubricant needs to be reduced at the prospective recording density of 4 Tb/in². In order to reduce a thickness of a film of a PFPE lubricant commonly used in the art, however, a molecular weight of the PFPE lubricant needs to be decreased. The smaller molecular weight of the PFPE lubricant has a problem that thermal stability is deteriorated. It has been understood that the above-described problems associated with reliability have not be sufficiently solved with common perfluoropolyether (PFPE)-based lubricants.

Particularly for a thin film magnetic recording medium having high surface smoothness, a novel lubricant is designed at a molecular level, and synthesized to solve the above-described trade-off. Moreover, there are a number of reports regarding lubricity of PFPE. As described, lubricants are very important in magnetic recording media.

Chemical structures of typical PFPE-based lubricants are depicted in Table 1.

TABLE 1
Fomblin-based lubricants

| | |
|---|---|
| | X—CF₂(OCF₂CF₂)n(OCF₂)mOCF₂—X(0.5<n/m<1) |
| Z | X ═ —OCF₃ |
| Z-DOL | X ═ —CH₂OH |
| Z-DIAC | X ═ —COOH |
| Z-Tetraol | 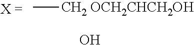 |
| AM2001 | 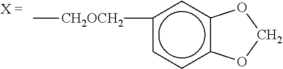 |

Other lubricants

| | |
|---|---|
| A20H | 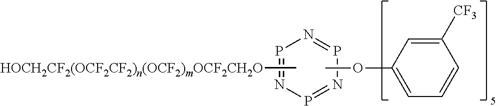 |
| Mono | F—(CF₂CF₂CF₂O)₁—CF₂CF₂CH₂—N(C₃H₇)₂ |

Z-DOL in Table 1 is one of lubricants typically used for thin-film magnetic recording media. Moreover, Z-tetraol (ZTMD) is a lubricant, in which a functional hydroxyl group is further introduced into a main chain of PFPE, and it has been reported that use of Z-tetraol enhances reliability of a drive while reducing a space at an interface between a head and a medium. It has been reported that A20H suppresses decomposition of the PFPE main chain with Lewis acid or Lewis base, and improves tribological properties. On the other hand, it has been reported that Mono has a different polymer main chain and different polar groups to those of the PFPE, and the polymer main chain and polar groups of Mono are respectively poly-n-propyloxy, and amine, and Mono reduces adhesion interactions at near contact.

However, a typical solid lubricant, which has a high melting point and is considered thermally stable, disturbs an electromagnetic conversion process that is extremely highly sensitive, and moreover, an abrasion powder scraped by a head is generated on a running track. Therefore, abrasion properties are deteriorated. As described above, the liquid lubricant has mobility that enables to move the adjacent lubricant layer to replenish the lubricant removed due to abrasion by the head. However, the lubricant is span-off from a surface of the disk especially at a high temperature during driving of the disk, because of the mobility of the lubricant, and thus the lubricant is reduced. As a result, a protection function is lost. Accordingly, a lubricant having a high viscosity and low volatility is suitably used, and use of such a lubricant enables to prolong a service life of a disk drive with suppressing an evaporation rate.

Considering the above-described lubricating systems, requirements for a low-friction and low-abrasion lubricant used for thin film magnetic recording media are as follows.
(1) Low volatility.
(2) Low surface tension for a surface filling function.
(3) Interaction between terminal polar groups and a surface of a disk.
(4) High thermal and oxidization stability in order to avoid decomposition or reduction over a service period.
(5) Chemically inactive with metals, glass, and polymers, and no abrasion powder generated from a head or a guide.
(6) No toxicity and no flammability.
(7) Excellent boundary lubricating properties.
(8) Soluble with organic solvents.

Recently, an ionic liquid has been attracted attentions as one of solvents for synthesis of organic or inorganic materials and being friendly to the environments in the fields of electricity storage materials, a separation technology, and a catalyst technology. The ionic liquid is roughly classified as a molten salt having a low melting point. The ionic liquid is typically a molten salt having a melting point of 100° C. or lower, among the above-mentioned molten salts. The important properties of the ionic liquid used as a lubricant are low volatility, inflammability, thermal stability, and an excellent dissolving performance.

For example, abrasion and wear of a surface of a metal or ceramic may be reduced by using a certain ionic liquid compared to a conventional hydrocarbon-based lubricant. For example, there is a report that, in the case where an imidazole cation-based ionic liquid is synthesized by substituting with a fluoroalkyl group, and tetrafluoroboric acid salt or hexafluorophosphoric acid salt of alkyl imidazolium is used for steel, aluminium, copper, single crystal SiO₂, silicon, or sialon ceramics (Si—Al—O—N), tribological properties more excellent than those of cyclic phosphazene (X-1P) or PFPE are exhibited. Moreover, there is a report that an ammonium-based ionic liquid reduces frictions more than a base oil in the region of elastohydrodynamic to boundary lubrication. Moreover, effects of the ionic liquid as an additive for a base oil have been studied, and a chemical or tribochemical reaction of the ionic liquid has been researched to understand lubricating systems. However, there are a few application examples of the ionic liquid to magnetic recording media. For example, Gong, X., Kozbial, A., Rose, F., Li, L., Effect of π−π+ Stacking on the Layering of Ionic Liquids Confined to an Amorphous Carbon Surface., Applied Mater. Interfaces 2015, vol. 7, pp. 7078-7081 reports an ionic liquid of an imidazole-based

tris(pentafluoroethyl)trifluorophosphate. This only suggests its possibility, and does not mention any specific tribological properties of it.

Among the ionic liquids, a perfluorooctanoic acid alkyl ammonium salt is a protic ionic liquid (PIL), and has been reported as having a significant effect of reducing frictions of magnetic recording media compared to Z-DOL mentioned above (for example, <1> Japanese Patent No. 2581090, <2> Japanese Patent No. 2629725, <3> Kondo, H., Seki, A., Watanabe, H., & Seto, J., (1990). Frictional is Properties of Novel Lubricants for Magnetic Thin Film Media, IEEE Trans. Magn. Vol. 26, No. 5, (September 1990), pp. 2691-2693, ISSN: 0018-9464, <4> Kondo, H., Seki, A., & Kita, A., (1994a). Comparison of an Amide and Amine Salt as Friction Modifiers for a Magnetic Thin Film Medium. Tribology Trans. Vol. 37, No. 1, (January 1994), pp. 99-104, ISSN: 0569-8197). However, the above-mentioned perfluorocarboxylic acid ammonium salts have weak interaction between a cation and an anion and hence thermal stability at a high temperature is deteriorated.

Meanwhile, the limit of a surface recording density of a hard disk is said to be from 1 Tb/in² to 2.5 Tb/in². Currently, the surface recording density is getting close to the limit, but developments of technology for increasing capacities have been actively conducted with a reduction in particle size of magnetic particles as a premise. As the technology for increasing capacities, there are a reduction in an effective flying height and introduction of Shingle Write (BMP).

Moreover, there is “thermally-assisted magnetic recording (heat assisted magnetic recording)” as the next-generation recording technology. The outline of the thermally-assisted magnetic recording is illustrated in FIG. 3. In FIG. 3, the referential numeral 1 is laser light, the referential numeral 2 is near field light, the referential numeral 3 is a recording head (PMR element), and the referential numeral 4 is a reproducing head (TMR element). The problems of the thermally-assisted magnetic recording include a deterioration of durability due to evaporation or deterioration of a lubricant on a surface of a magnetic layer because a recording area is heated by laser during recording and reproducing. Even though it is a short period, there is a possibility that a thin film magnetic recording medium is exposed to a high temperature, which is 400° C. or higher, in thermally-assisted magnetic recording. Therefore, there are concerns about thermal stability of a lubricant generally used for thin film magnetic recording media, such as Z-DOL and Z-TETRAOL.

Here, Watanabe et al. has reported that proton transfer and thermal stability of a protic ionic liquid largely depend on ΔpKa, and thermal stability of the ionic liquid is significantly improved by using the acid with which the ΔpKa is 15 or greater when DBU (1,8-diazabicyclo[5,4,0]undec-7-ene) is used as a base (see, for example, Miran, M. S., Kinoshita, H., Yasuda, T., Susan, M. A. B. H., Watanabe, M., Physicochemical Properties Determined by ΔpKa for Protic Ionic Liquids Based on an Organic Super-strong Base with Various Bronsted Acids, Phys. Chem. Chem. Phys., Vol. 14, pp. 5178-5186 (2012)). Moreover, Kondo et al. have reported that a perfluorooctanesulfonic acid octadecyl ammonium salt-based protic ionic liquid having large ΔpKa improves durability of a magnetic recording medium (see, for example, <1>Hirofumi Kondo, Makiya Ito, Koki Hatsuda, Kyung Sung Yun and Masayoshi Watanabe, “Novel Ionic Lubricants for Magnetic Thin Film” Media, IEEE TRANSACTIONS ON MAGNETICS, VOL. 49, NO. 7, pp. 3756-3759, JULY (2013), <2>International Publication No. WO2014/104342). Regarding thermal resistance of ionic liquid, however, a relationship between a molecular structure constituting the pyrrolidinium-based ionic liquid and physical or chemical characteristics has not been fully understood yet. A combination of a cation and an anion largely influences on physical or chemical characteristics of an ionic liquid.

A variety of the anion site is many, but the relationship is not clear unless the cation is a cation structurally similar to the anion (see, for example, Dzyuba, S. V.; Bartsch, R. A., “Influence of Structural Variations in

1-Alkyl(aralkyl)-3-Methylimidazolium Hexafluorophosphates and Bis(trifluoromethylsulfonyl)imides on Physical Properties of the Ionic Liquids, Chem. Phys. Phys. Chem. 2002, 3, 161-160. For example, viscosity of the liquid increases, as hydrogen bonding strength of halogen is stronger (Cl>Br >I). However, the method for increasing the viscosity is not limited to the increase in the hydrogen bonding strength. For example, the viscosity can be increased by is varying an alkyl chain of imidazole. Similarly, the combination of the anion and cation influences on a melting point, surface tension, and thermal stability, but a wide range of researches has not be conducted on an effect of the anion. Accordingly, it is possible to change physical or chemical characteristics of an ionic liquid by with a combination of cations or anions, but it is difficult to predict.

Considering applications to hard disks, there is a need for solubility to fluorine-based solvents used in production lines (e.g., special solvent Vertrel available from Du Pont Co.) like in commercially available perfluoropolyethers. Note that, when using fluorine-based solvents, production lines do not need to be explosion proof. Thus, fluorine-based solvents have suitably been used as a solvent used for lubricants in production lines of hard discs. Compounds other than perfluoropolyether-based compounds are not very good in solubility to solubility to fluorine-based solvents. As a result, their applications to hard discs have been limited despite their good lubricating properties.

Also, conventionally-known lubricants that are used are long-chain fatty acid or ester thereof. When the ionic liquid is used in combination with them, the ionic liquid needs to be excellent in solubility to the hydrocarbon-based solvents used in the lubricants.

### SUMMARY OF THE INVENTION

The present invention is proposed based on the above-described situations in the art, and provides an ionic liquid having excellent solubility to fluorine-based solvents and hydrocarbon-based solvents and excellent lubricity even at high temperatures, a lubricant having excellent lubricity even at high temperatures and excellent suitability to production lines of magnetic recording media, and a magnetic recording medium having excellent practical properties.

<122 A lubricant including:

an ionic liquid including a conjugate base and a conjugate acid, wherein the conjugate acid includes a straight-chain hydrocarbon group having 6 or greater carbon atoms and a straight-chain hydrocarbon group having 6 or greater but 14 or less carbon atoms, and
wherein a pKa of an acid that is a source of the conjugate base in acetonitrile is 10 or less.

<2> The lubricant according to <1>,
wherein the conjugate acid is represented by General Formula (A) below,

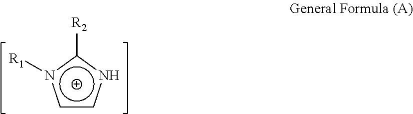

where, in General Formula (A),
R₁ is a group including a straight-chain hydrocarbon group having 6 or greater carbon atoms and R₂ is a group including a straight-chain hydrocarbon group having 6 or greater but 14 or less carbon atoms; or
R₁ is a group including a straight-chain hydrocarbon group having 6 or greater but 14 or less carbon atoms and R₂ is a group including a straight-chain hydrocarbon group having 6 or greater carbon atoms.

<3> The lubricant according to <1> or <2>,
wherein the conjugate base is represented by General Formula (X) or General Formula (Y) below,

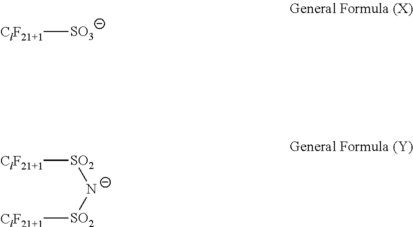

where, in General Formula (X), 1 is an integer of 1 or greater but 12 or less, and
where, in General Formula (Y), 1 is an integer of 1 or greater but 12 or less.

<4> A magnetic recording medium including:

a non-magnetic support;

a magnetic layer disposed on the non-magnetic support; and

the lubricant according to any one of <1> to <3>, disposed on the magnetic layer.

<5> An ionic liquid including:

a conjugate base; and

a conjugate acid,
wherein the conjugate acid includes a straight-chain hydrocarbon group having 6 or greater carbon atoms and a straight-chain hydrocarbon group having 6 or greater but 14 or less carbon atoms, and
wherein a pKa of an acid that is a source of the conjugate base in acetonitrile is 10 or less.

<6> The ionic liquid according to <5>,
wherein the conjugate acid is represented by General Formula (A) below,

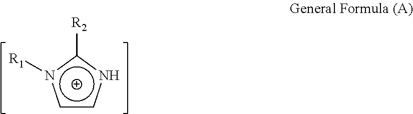

where, in General Formula (A),
R₁ is a group including a straight-chain hydrocarbon group having 6 or greater carbon atoms and R₂ is a group including a straight-chain hydrocarbon group having 6 or greater but 14 or less carbon atoms; or
R₁ is a group including a straight-chain hydrocarbon group having 6 or greater but 14 or less carbon atoms and R₂ is a group including a straight-chain hydrocarbon group having 6 or greater carbon atoms.

<7> The ionic liquid according to <5> or <6>,
wherein the conjugate base is represented by General Formula (X) or General Formula (Y) below,

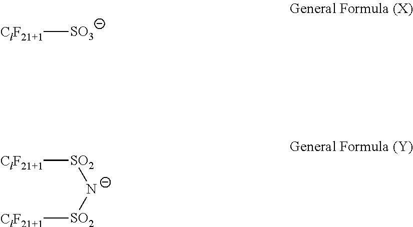

where, in General Formula (X), 1 is an integer of 1 or greater but 12 or less, and
where, in General Formula (Y), 1 is an integer of 1 or greater but 12 or less.

The present invention can provide an ionic liquid having excellent solubility to fluorine-based solvents and hydrocarbon-based solvents and excellent lubricity even at high temperatures, a lubricant having excellent lubricity even at high temperatures and excellent suitability to production lines of magnetic recording media, and a magnetic recording medium having excellent practical properties.

### BRIEF DESCRIPTION OF THE DRAWINGS

FIG. 1 is a graph presenting a transition and prediction of an in-plane recording density of a hard disk drive;

FIG. 2 is a road map of a head media spacing (HMS) relative to an in-plane recording density of a hard disk;

FIG. 3 is a schematic view illustrating thermally-assisted magnetic recording;

FIG. 4 is a cross-sectional view illustrating one example of a hard disk according to one embodiment of the present invention; and

FIG. 5 is a cross-sectional view illustrating one example of a magnetic tape according to one embodiment of the present invention.

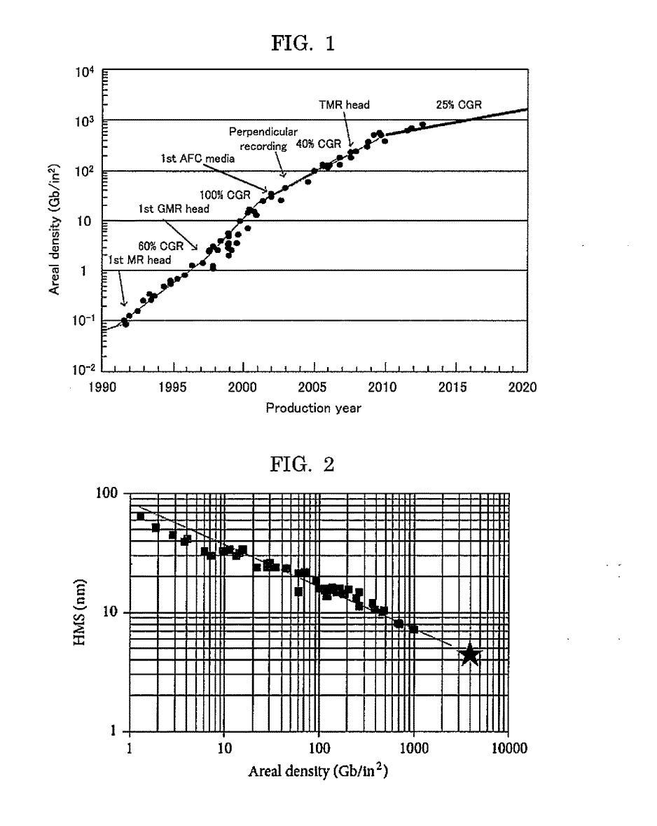

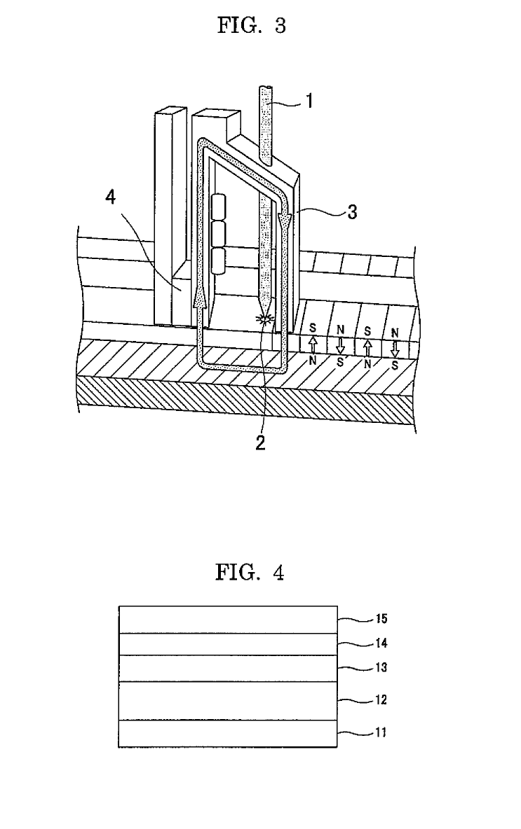

### DETAILED DESCRIPTION OF THE INVENTION

Embodiments of the present invention are specifically described with reference to drawing hereinafter in the following order.
1. Lubricant and ionic liquid
2. Magnetic recording medium
3. Examples
<1. Lubricant and ionic liquid>

A lubricant described as one embodiment of the present invention includes an ionic liquid including a conjugate acid and a conjugate base.

An ionic liquid described as one embodiment of the present invention includes a conjugate acid and a conjugate base.

In the ionic liquid, the conjugate acid includes a straight-chain hydrocarbon group having 6 or greater carbon atoms and a straight-chain hydrocarbon group having 6 or greater but 14 or less carbon atoms.

In the ionic liquid, a pKa of an acid that is a source of the conjugate base in acetonitrile is 10 or less.

The ionic liquid in the present embodiment includes a conjugate acid and a conjugate base, where a pKa of an acid that is a source of the conjugate base in acetonitrile is 10 or less. Thus, the bond between the ions is strong, which makes it possible for the ionic liquid to exhibit excellent thermal stability. The ionic liquid has two or more groups each including a straight-chain hydrocarbon group having 6 or greater carbon atoms in the cation portion and the number of carbon atoms in one of the groups is 14 or less, and thus the ionic liquid also has excellent lubricating properties. In addition, the ionic liquid has been improved in solubility to fluorine-based solvents and hydrocarbon-based solvents. Since solubility is improved to CF₃(CHF)₂CF₂CF₃ that is often used as a fluorine-based solvent in a lubricant applying step of hard discs, production lines of magnetic recording media do not need to be explosion proof, as a result. Considering the fact that the materials widely used as lubricants are long-chain fatty acid or ester thereof, improved solubility to hydrocarbon-based solvents results in improved compatibility, which means that the ionic liquid can exhibit its effects as an additive. Also, it has an effect of reducing the melting point and thus has a wider application field as a lubricant.

Here, a lubricant containing an ionic liquid is generally used in a state where the concentration of the ionic liquid is about 0.05% by mass. Therefore, solubility of the ionic liquid to fluorine-based solvents needs to be 0.05% by mass or more. Also, depending on the situations where it is used, more solubility may be required. Furthermore, considering, for example, changes in use situations and storage conditions, solubility of 0.1% by mass or more is required [i.e., the ionic liquid is 0.1 parts by mass or more relative to 100 parts by mass of CF₃(CHF)₂CF₂CF_{3].}

The pKa is 10 or less, which is a strong acid, and is preferably 6.0 or less.

The lower limit of the pKa is not particularly limited and may be appropriately selected depending on the intended purpose, but the pKa is preferably−5.0 or greater.

In the present specification, “pKa” is an acid dissociation constant, and is an acid dissociation constant in acetonitrile.

### <<Conjugate Base>>

The conjugate base is not particularly limited and may be appropriately selected depending on the intended purpose, as long as pKa of an acid that is a source of the conjugate base in acetonitrile is 10 or less. Examples of the conjugate base include a conjugate base represented by General Formula (X) below, a is conjugate base represented by General Formula (Y) below, a conjugate base represented by General Formula (U) below, a conjugate base represented by General Formula (V) below, and a conjugate base represented by General Formula (W) below. Among the above-listed examples, a conjugate base represented by General Formula (X) below and a conjugate base represented by General Formula (Y) below are preferable because solubility of the ionic liquid to a solvent becomes high.

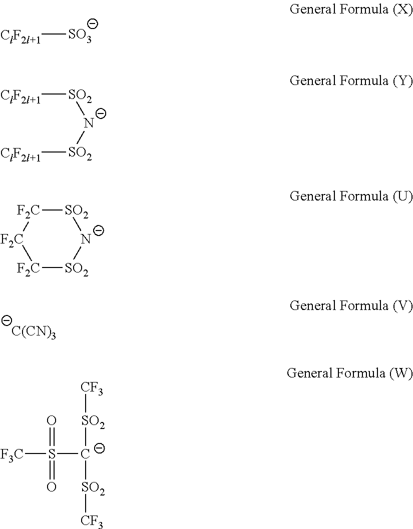

In General Formula (X), 1 is an integer of 1 or greater but 12 or less, and is preferably an integer of 1 or greater but 6 or less.

In General Formula (Y), 1 is an integer of 1 or greater but 12 or less, and is preferably an integer of 1 or greater but 6 or less.

As the acid that is a source of the conjugate base (HA), Bronsted acids regarded as super acid, such as bis((perfluoroalkyl)sulfonyl)imide [(C₁F₂ₗ₊₁SO₂)₂NH](pKa=0 to 0.3), perfluorocyclopropane sulfoimide (pKa=−0.8), perfluoroalkyl sulfonic acid (CₘF₂ₘ₊₁SO₃H) (pKa=0.7), tris(perfluoroalkanesulfonyl)methide compounds [(CF₃SO₂)₃CH] (pKa=tricyanomethane (pKa=5.1), inorganic acids [e.g., nitric acid (pKa =9.4) and sulfuric acid (pKa=8.7)], tetrafluoroboric acid (pKa=1.8), and hexafluorophosphate, are preferable. The pKa of the above-listed acids are introduced, for example, in Non-Patent Literature: J. Org. Chem. Vol. 76, No. 2, p. 394.

### <<Conjugate Acid>>

The conjugate acid has two or more straight-chain hydrocarbon groups each having 6 or greater carbon atoms and the number of carbon atoms in one of the groups is 14 or less. The number of carbon atoms in the hydrocarbon group, which may include a partially fluorinated carbon, is not particularly limited and may be appropriately selected depending on the intended purpose so long as it is 6 or greater in total. However, it is preferably 10 or greater.

The upper limit of the number of carbon atoms in the straight-chain hydrocarbon group having 6 or greater carbon atoms is not particularly limited and may be appropriately selected depending on the intended purpose. The upper limit of the number of carbon atoms is preferably 30 or less, more preferably 25 or less, and particularly preferably 20 or less in view of availability of raw materials. When the hydrocarbon group has a long chain, lubricity can be improved, with reducing a coefficient of friction.

The group including a straight-chain hydrocarbon group having 6 or greater carbon atoms may be branched but is preferably the straight-chain hydrocarbon group having 6 or greater carbon atoms. It may include a partially fluorinated carbon. Since the solubility to solvents tends to be low when the number of carbon atoms is large, the number of carbon atoms of the hydrocarbon group is determined in view of both an effect of reducing a coefficient of friction, and solubility to solvents.

Similarly, the straight-chain hydrocarbon group having 6 or greater but 14 or less carbon atoms may be branched but is preferably the straight-chain hydrocarbon group having 6 or greater carbon atoms. It may include a partially fluorinated carbon.

The conjugate acid is preferably represented by General Formula (A) below.

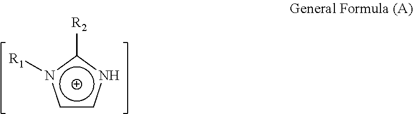

In General Formula (A), R₁ is a group including a straight-chain hydrocarbon group having 6 or greater carbon atoms and R₂ is a group including a straight-chain hydrocarbon group having 6 or greater but 14 or less carbon atoms; s or R₁ is a group including a straight-chain hydrocarbon group having 6 or greater but 14 or less carbon atoms and R₂ is a group including a straight-chain hydrocarbon group having 6 or greater carbon atoms.

### <<Preferable Examples of Ionic Liquid>>

Preferable examples of the ionic liquid are an ionic liquid represented by General Formula (1) below.

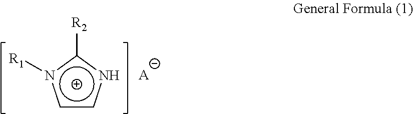

In General Formula (1), R₁ is a group including a straight-chain hydrocarbon group having 6 or greater carbon atoms and R₂ is a group including a straight-chain hydrocarbon group having 6 or greater but 14 or less carbon atoms; or R₁ is a group including a straight-chain hydrocarbon group having 6 or greater but 14 or less carbon atoms and R₂ is a group including a straight-chain hydrocarbon group having 6 or greater carbon atoms. A⁻ is a conjugate base.

The ionic liquid represented by General Formula (1) above is preferably an ionic liquid represented by General Formula (1-1) below and an ionic liquid represented by General Formula (1-2) below.

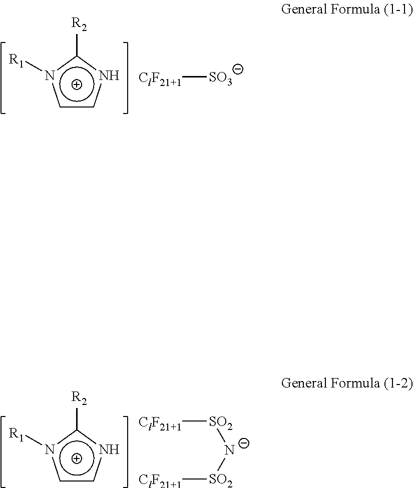

In General Formula (1-1), R₁ is a group including a straight-chain hydrocarbon group having 6 or greater carbon atoms and R₂ is a group including a straight-chain hydrocarbon group having 6 or greater but 14 or less carbon atoms; or R₁ is a group including a straight-chain hydrocarbon group having 6 or greater but 14 or less carbon atoms and R₂ is a group including a straight-chain hydrocarbon group having 6 or greater carbon atoms. 1 is an integer of 1 or greater but 12 or less.

In General Formula (1-2), R₁ is a group including a straight-chain hydrocarbon group having 6 or greater carbon atoms and R₂ is a group including a straight-chain hydrocarbon group having 6 or greater but 14 or less carbon atoms; or R₁ is a group including a straight-chain hydrocarbon group having 6 or greater but 14 or less carbon atoms and R₂ is a group including a straight-chain hydrocarbon group having 6 or greater carbon atoms. 1 is an integer of 1 or greater but 12 or less.

A synthesis method of the ionic liquid is not particularly limited and may be appropriately selected depending on the intended purpose. For example, various types of the ionic liquid can be synthesized with reference to the method disclosed in Examples below.

The lubricant of the present embodiment may use the above-described ionic liquid alone or in combination with a conventional lubricant. Examples of the lubricant used in combination include long-chain carboxylic acid, long-chain carboxylic acid ester, perfluoroalkyl carboxylic acid ester, perfluoroalkyl carboxylate, perfluoroalkyl perfluoroalkyl carboxylate, and a perfluoropolyether derivative.

Moreover, an extreme pressure agent may be used in combination at a blending ratio of about 30:70 to about 70:30 in a mass ratio in order to maintain a lubricating effect under severe conditions. The extreme pressure agent reacts with a surface of a metal with friction heat generated when the lubricant is partially in contact with the metal in a boundary lubrication region, and forms a coating film of a reaction product. As a result, friction and abrasion are prevented. As the extreme pressure agent, for example, any of a phosphorus-based extreme pressure agent, a sulfur-based extreme pressure agent, a halogen-based extreme pressure agent, an organic metal-based extreme pressure agent, or a complex extreme pressure agent can be used.

Moreover, an anti-rust agent may be optionally used in combination. The anti-rust agent may be any anti-rust agent typically used for this kind of magnetic recording media. Examples of the anti-rust agent include phenols, naphthols, quinones, heterocyclic compounds containing a nitrogen atom, heterocyclic compounds containing an oxygen atom, and heterocyclic compounds containing a sulfur atom. Moreover, the anti-rust agent may be mixed with the lubricant. Alternatively, the anti-rust agent and the lubricant may be deposited as two or more layers by forming a magnetic layer on a non-magnetic support, and applying an anti-rust agent layer on the upper part of the magnetic layer, followed by applying a lubricant layer.

As a solvent of the lubricant, for example, a single use or a combination of alcoholic solvents, such as isopropyl alcohol (IPA), and ethanol, can be used. For example, a mixture of a hydrocarbon-based solvent, such as normal-hexane, and a fluorine-based solvent can be used.

The solvent is preferably a fluorine-based solvent. Examples of the fluorine-based solvent include hydrofluoroethers [e.g., C₃F₇OCH_{3,} C₄F₉OCH_{3,} C₄F₉OC₂H₅, C₂F₅CF(OCH₃) C₃F₇, and CF₃(CHF)₂CF₂CF₃]. The fluorine-based solvent may be used as a mixture with alcohol, such as IPA, ethanol, and methanol.

The fluorine-based solvent may be a commercially available product. Examples of the commercially available product include: Novec™ 7000, 7100, 7200, 7300, and 71IPA available from 3M Company; and Vertrel XF, and X-P10 available from Du Pont-Mitsui Fluorochemicals Company, Ltd.

### <2. Magnetic Recording Medium>

Next, a magnetic recording medium using the above-described lubricant is described. A magnetic recording medium described as one embodiment of the present invention includes at least a magnetic layer on a non-magnetic support, and the above-described lubricant is held on the magnetic layer.

The lubricant of the present embodiment can be applied for so-called a thin film-metal-type magnetic recording medium, in which a magnetic layer formed on a non-magnetic support by a method, such as vapor deposition and sputtering. Moreover, the lubricant can be also applied for a magnetic recording medium having a structure, in which a base layer is disposed between a non-magnetic support and a magnetic layer. Examples of such a magnetic recording medium include a magnetic disk, and a magnetic tape.

FIG. 4 is a cross-sectional view illustrating one example of a hard disk. The hard disk has a structure, in which a substrate 11, a base layer 12, a magnetic layer 13, a protective carbon layer 14, and a lubricant layer 15 are sequentially laminated.

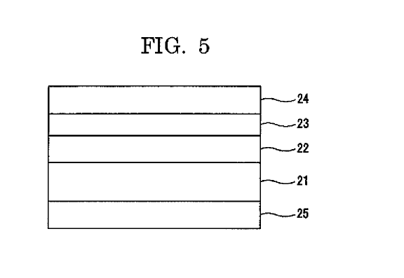

Moreover, FIG. 5 is a cross-sectional view illustrating one example of a magnetic tape. The magnetic tape has a structure, in which a back-coating layer 25, a substrate 21, a magnetic layer 22, a protective carbon layer 23, and a lubricant layer 24 are sequentially laminated.

In the magnetic disk illustrated in FIG. 4, each of the substrate 11 and the base layer 12 corresponds to the non-magnetic support. In the magnetic tape illustrated in FIG. 5, the substrate 21 corresponds to the non-magnetic support. In the case where a rigid substrate, such as an Al alloy plate, and a glass plate, is used as the non-magnetic support, a surface of the substrate may be made hard by forming an oxidized film, such as anodizing or a Ni—P coating on the surface of the substrate.

Each of the magnetic layers 13 and 22 is formed as a continuous film by a method, such as plating, sputtering, vacuum deposition, and plasma CVD. Examples of the magnetic layers 13 and 22 include: longitudinal magnetic recording metal magnetic films formed of metals (e.g., Fe, Co, and Ni), Co—Ni-based alloys, Co—Pt-based alloys, Co—Ni—Pt-based alloys, Fe—Co-based alloys, Fe—Ni-based alloys, Fe—Co—Ni-based alloys, Fe—Ni—B-based alloys, Fe—Co—B-based alloys, or Fe—Co—Ni—B-based alloys; and perpendicular magnetic recording metal magnetic thin films, such as Co—Cr-based alloy thin films, and Co—O-based thin films.

In the case where a longitudinal magnetic recording metal magnetic thin film is formed, particularly, a non-magnetic material, such as Bi, Sb, Pb, Sn, Ga, In, Ge, Si, and T1, is formed as a base layer 12 on a non-magnetic support in advance, and a metal magnetic material is deposited through vapor deposition or sputtering in a perpendicular direction to diffuse the non-magnetic material into the magnetic metal thin film, to thereby improve a coercive force as well as eliminating orientation to assure in-plane isotropy.

Moreover, a hard protective layer 14 or 23, such as a carbon film, a diamond-formed carbon film, a chromium oxide film, and SiO₂ film, may be formed on a surface of the magnetic layer 13 or 22.

Examples of a method for applying the above-mentioned lubricant to such a metal thin film magnetic recording medium include a method for top-coating a surface of the magnetic layer 13 or 22, or a surface of the protective layer 14 or 23 with the lubricant, as illustrated in FIGS. 2 and 5. A coating amount of the s lubricant is preferably from 0.1 mg/m² to 100 mg/m², more preferably from 0.5 mg/m² to 30 mg/m², and particularly preferably from 0.5 mg/m² to 20 mg/m².

As illustrated in FIG. 5, moreover, a metal thin film magnetic tape may optionally have a back-coating layer 25, other than a metal magnetic thin film, which is the magnetic layer 22.

The back-coating layer 25 is formed by adding a carbon-based powder for imparting conductivity, or an inorganic pigment for controlling a surface roughness to a resin binder, and applying the resin binder mixture. In the present embodiment, the above-described lubricant may be internally added to the back-coating layer 25, or applied to the back-coating layer 25 as top coating. Moreover, the above-described lubricant may be internally added to both the magnetic layer 22 and the back-coating layer 25, or applied to both the magnetic layer 22 and the back-coating layer 25 as top coating.

As another embodiment, moreover, the lubricant can be applied for a so-called coating-type magnetic recording medium, in which a magnetic coating film is formed as a magnetic layer by applying a magnetic coating material onto a surface of a non-magnetic support. In the coating-type magnetic recording medium, the non-magnetic support, a magnetic powder constituting the magnetic coating film, and the resin binder for use can be selected from any of those known in the art.

Examples of the non-magnetic support include: polymer substrates formed of polymer materials, such as polyesters, polyolefins, cellulose derivatives, vinyl-based resins, polyimides, polyamides, and polycarbonate; metal substrates formed of aluminium alloys, or titanium alloys; ceramic substrates formed of alumina glass; and glass substrates. Moreover, a shape of the non-magnetic support is not particularly limited, and may be any form, such as a tape, a sheet, and a drum. Furthermore, the non-magnetic support may be subjected to a surface treatment to form fine irregularities in order to control surface properties of the non-magnetic support.

Examples of the magnetic powder include: ferromagnetic iron oxide-based particles, such as γ-Fe₂O₃, cobalt-coated γ-Fe₂O₃; ferromagnetic chromium dioxide; ferromagnetic metal-based particles formed of a metal, such as Fe, Co, and Ni, or an alloy containing any of the above-listed metals; and hexagonal ferrite particles in the form of hexagonal plates.

Examples of the resin binder include: polymers, such as vinyl chloride, vinyl acetate, vinyl alcohol, vinylidene chloride, acrylic acid ester, methacrylic acid ester, styrene, butadiene, and acrylonitrile; copolymers combining two or more selected from the above-listed polymers; polyurethane resins; polyester resins; and epoxy resins. In order to improve dispersibility of the magnetic powder, a hydrophilic polar group, such as a carboxylic acid group, a carboxyl group, and a phosphoric acid group, may be introduced into any of the above-listed binders.

Other than the magnetic powder and the resin binder, additives, such as a dispersing agent, an abrasive, an antistatic agent, and an anti-rust agent, may be added to the magnetic coating film.

As a method for retaining the above-described lubricant in the coating-type magnetic recording medium, there are a method where the lubricant is internally added to the magnetic layer constituting the magnetic coating film formed on the non-magnetic support, a method where the lubricant is applied on a surface of the magnetic layer as top coating, and a combination of the above-listed methods. In the case where the lubricant is internally added into the magnetic coating film, the lubricant is added in an amount of from 0.2 parts by mass to 20 parts by mass relative to 100 parts by mass of the resin binder.

In the case where a surface of the magnetic layer is top-coated with the lubricant, moreover, a coating amount of the lubricant is preferably from 0.1 mg/m² to 100 mg/m², and more preferably from 0.5 mg/m² to 20 mg/m². A possible deposition method in the case where the lubricant is applied as top coating is that the ionic liquid is dissolved in a solvent, and the obtained solution is applied or sprayed, or a magnetic recording medium is dipped in the solution.

The magnetic recording medium, to which the lubricant of the present embodiment is applied, exhibits excellent running performances, abrasion resistance, and durability because of a lubricating effect, and can further improve thermal stability.

### EXAMPLES

### 3. Examples

Specific examples of the present invention are explained below. In the examples, ionic liquids were synthesized, and lubricants including the ionic liquids were produced. Then, solubility of the ionic liquids to Vertrel [CF₃(CHF)₂CF₂CF₃], which was a fluorine-based solvent were determined, and to n-hexane, which was a hydrocarbon-based solvent. Then, the lubricant solutions were applied surfaces of the magnetic disks and magnetic tapes and durability of each disk and durability of each tape were evaluated. Production of a magnetic disk, a durability test of the disk, production of a magnetic tape, and a durability test of the tape were performed in the following manner. Note that, the present invention is not limited to these examples.

<Production of magnetic disk>

A magnetic thin film was formed on a glass substrate to produce a magnetic disk as illustrated in FIG. 4, for example, according to International Publication No. WO2005/068589. Specifically, a chemically reinforced glass disk, which was formed of aluminium silicate glass and had an outer diameter of 65 mm, an inner diameter of 20 mm, and a disk thickness of 0.635 mm, was prepared, and a surface of the glass disk was polished so that Rmax of the surface was to be 4.8 nm, and Ra of the surface was to be 0.43 nm. The glass substrate was subjected to ultrasonic cleaning for 5 minutes each in pure water and in isopropyl alcohol (IPA) having the purity of 99.9% or greater, and the washed glass substrate was left to stand in saturated IPA steam for 1.5 minutes, followed by drying. The resultant glass substrate was provided as a substrate 11.

On the substrate 11, a NiAl alloy (Ni: 50 mol %, Al: 50 mol %) thin film in the thickness of 30 nm as a seed layer, a CrMo alloy (Cr: 80 mol %, Mo: 20 mol %) thin film in the thickness of 8 nm as a base layer 12, and a CoCrPtB alloy (Co: 62 mol %, Cr: 20 mol %, Pt: 12 mol %, B: 6 mol %) thin film in the thickness of 15 nm as a magnetic layer 13 were sequentially formed by DC magnetron sputtering.

Subsequently, a 5 nm-thick protective carbon layer 14 formed of amorphous diamond-like carbon was formed by plasma CVD, and the resultant disk sample was subjected to ultrasonic cleaning for 10 minutes in isopropyl alcohol (IPA) having the purity of 99.9% or greater inside a cleaner to remove impurities on a surface of the disk, followed by drying. Thereafter, a n-hexane/ethanol mixed solution of an ionic liquid was applied on a surface of the disk by dip coating in the environment of 25° C. and 50% in relative humidity (RH), to form about 1 nm of a lubricant layer 15.

### <Measurement of Thermal Stability>

In the TG/DTA measurement, the measurement was performed by means of EXSTAR6000 available from Seiko Instruments Inc. at a temperature range of from 30° C. to 600° C. at a heating rate of 10° C./min with introducing air at a flow rate of 200 mL/min.

The endothermic peak temperature in the measurement was defined as a melting point.

### <Disk Durability Test>

A CSS durability test was performed by means of a commercially available strain-gauge-type disk friction-abrasion tester in the following manner. A hard disk was mounted on a rotatable spindle with tightening torque of 14.7 Ncm. Thereafter, a head slider was attached on the hard disk in a manner that a center of an air bearing surface at the inner circumference side of the head slider relative to the hard disk was 17.5 mm from a center of the hard disk. The head used for the measurement was an IBM3370-type inline head, a material of the slider was Al₂O₃—TiC, and the head load was 63.7 mN. In the test, the maximum value of friction force was monitored per CSS (contact, start, and stop) in the environment of 100 in cleanliness, 25° C., and 60% RH. The number of times when a coefficient of is friction was greater than 1.0 was determined as a result of the CSS durability test. When a result of the CSS durability test was greater than 50,000, the result was represented as “>50,000.” Moreover, a CSS durability test was similarly performed after performing a heating test for 3 minutes at a temperature of 300° C., in order to study heat resistance.

### <Production of Magnetic Tape>

A magnetic tape having a cross-sectional structure as illustrated in FIG. 5 was produced. First, Co was deposited on a substrate 21 formed of a 5 μm-thick MICTRON (aromatic polyamide) film available from TORAY INDUSTRIES, INC. by oblique deposition to form a magnetic layer 22 formed of a ferromagnetic metal thin film having a film thickness 100 nm. Next, a protective carbon layer 23 formed of a 10 nm-thick diamond-like carbon was formed on a surface of the ferromagnetic metal thin film by plasma CVD, followed by cutting the resultant into a strip having a width of 6 mm. An ionic liquid dissolved in IPA was applied onto the protective carbon layer 23 in a manner that a film thickness of the ionic liquid solution was about 1 nm. In this manner, a lubricant layer 24 is formed on the magnetic layer to thereby produce a sample tape.

<Tape Durability test>

Each sample tape was subjected to a measurement of still durability in an environment having a temperature of −5° C. and in an environment having a temperature of 40° C. and 30% RH, and measurements of a coefficient of friction and shuttle durability in an environment having a temperature of −5° C. and in an environment having a temperature of 40° C. and 90% RH. The still durability was evaluated by a decay time of an output in a paused state decayed by −3 dB. The shuttle resistant was evaluated by the number of shuttles taken until an output was reduced by 3 dB when repeated shuttle run was performed for 2 minutes per time. Moreover, a durability test was similarly performed after performing a heating test for 10 minutes at a temperature of 100° C., in order to study heat resistance.

The ionic liquid in the present embodiment includes a conjugate base and a conjugate acid, where a pKa of an acid that is a source of the conjugate base in acetonitrile is 10 or less. Moreover, the ionic liquid has, in the conjugate acid (the cation portion), two or more hydrocarbon groups each having 6 or greater carbon atoms, where the number of carbon atoms in one of the groups is preferably 14 or less. Thermal stability of the ionic liquid and durability of magnetic recording media using the ionic liquid were studied. In addition, solubility to hydrocarbon-based solvents and fluorine-based solvents was also studied.

In the present specification, the measurement of FTIR was performed by means of FT/IR-460 available from JASCO Corporation according to a transmission method using KBr plates or KBr pellets. The resolution of the measurement was 4 cm⁻¹.

The ¹H-NMR and ¹³C-NMR spectra were measured by means of Varian Mercury Plus 300 nuclear magnetic resonance spectrometer (available from Varian, Inc.). A chemical shift of ¹HNMR was represented with ppm comparing with an internal standard (TMS or deuterated solvent peak at 0 ppm). Splitting patterns were presented by denoting a singlet as s, a doublet as d, a triplet as t, a quartet as q, a quintet as quint, a multiplet as m, and a broad peak as br.

### Example 1A

### Synthesis of nonafluorobutanesulfonic acid-1-dodecyl-2-undecylimidazolium

A synthesis of nonafluorobutanesulfonic acid-1-dodecyl-2-undecylimidazolium was performed according to the following scheme.

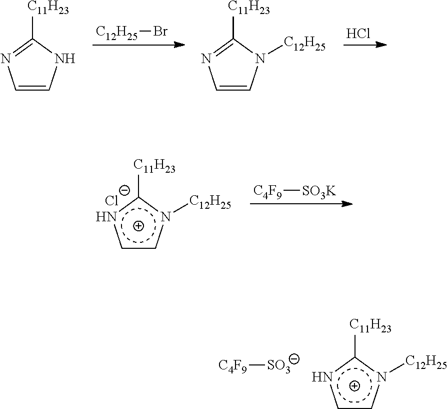

A flask was charged with 17.76 g of 2-undecylimidazole, 20.00 g of dodecyl bromide, 7.75 g of potassium hydroxide, and the mixture was heated in toluene under reflux for 8.0 hours. After returning the temperature to room temperature, the solvent was removed. The resultant was purified by silica gel column chromatography using a mixed solvent of n-hexane and ethyl acetate [n-hexane: ethyl acetate (volume ratio)=9:1] as an eluent. A purity of 27.35 g of 1-dodecyl-2-undecylimidazole, which was a colorless liquid, as measured by gas chromatography was 98.8% or greater. The yield was 87.7%.

In ethanol, 9.04 g of 1-dodecyl-2-undecylimidazole was dissolved. To the resultant solution, 3.18 g of an ethanol solution of 35% concentrated hydrochloric acid was added. Colorless crystals after removal of the solvent were dissolved in dichloromethane, and the resultant solution was washed with water until the wash liquid became neutral. After removal of the organic solvent, the resultant was recrystallized from a mixed solvent of n-hexane and ethyl acetate, to thereby obtain 8.30 g of colorless crystals of 1-dodecyl-2-undecylimidazolium chloride. The yield was 84.0%.

2.67 g of 1-dodecyl-2-undecylimidazolium chloride was dissolved in a mixed solvent of water and ethanol with heating, and an aqueous solution of 2.57 g of potassium nonafluorobutanesulfonate was added thereto. The resultant was heated under reflux for 1 hour. After cooling, the resultant was separated into an aqueous layer and an organic layer. The aqueous layer was extracted with dichloromethane, and the combined organic layer was washed until a result of the AgNO₃ test became negative. After drying the organic layer with anhydrous sodium sulfate, the solvent was removed to thereby obtain 4.88 g of colorless crystals of nonafluorobutanesulfonic acid-1-dodecyl-2-undecylimidazolium. The yield was 93.1%.

FTIR absorbance peaks of the product are presented below.

Absorbing vibrations were observed at 1,135 cm⁻¹, 1,235 cm⁻¹, 1,354 cm⁻¹, 1,470 cm⁻¹, 1,522 cm⁻¹, 2,853 cm⁻¹, 2,920 cm⁻¹, 3,081 cm⁻¹, and 3,147 cm⁻¹.

Peaks of the proton (¹H)NMR and carbon (¹³C)NMR of the obtained compound in CDCl₃ are presented below.

¹H-NMR(CDCl₃, δppm);0.856(t/J=6.8 Hz,6H), 1.180-1.400(m,34H), 1.720-1.860(m,4H), 2.934(t/J=8.0 Hz,2H), 3.988(t/J=7.8 Hz,2H), 7.048-7.057(m,1H), 7.291-7.302(m,1H), 13.710(brs,1H)

¹³C-NMR(CDCl₃, δppm);14.070, 22.647, 24.659, 26.347, 27.342, 28.943, 29.048, 29.288, 29.335, 29.441, 29.546, 30.093, 31.865, 47.734, 119.250, 122.410, 147.347

The product was determined as nonafluorobutanesulfonic acid-1-dodecyl-2-undecylimidazolium from the spectra above.

Note that, a pKa of an acid (nonafluorobutanesulfonic acid) that was a source of the conjugate base in the nonafluorobutanesulfonic acid-1-dodecyl-2-undecylimidazolium was 0.7 in acetonitrile.

### Example 2A

### Synthesis of bis(nonafluorobutanesulfonyl)imide-1-dodecyl-2-undecylimidazolium

A synthesis of bis(nonafluorobutanesulfonyl)imide-1-dodecyl-2-undecylimidazolium was performed according to the following scheme.

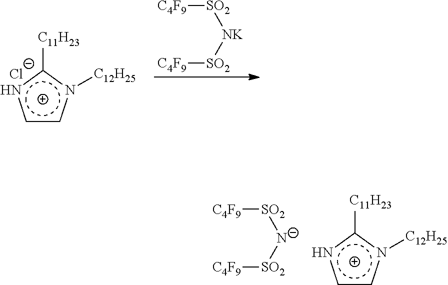

2.67 g of 1-dodecyl-2-undecylimidazolium chloride synthesized in Example 1A was dissolved in heated pure water. To the resultant solution, a solution prepared by dissolving 3.88 g of potassium bis(nonafluorobutanesulfonyl)imide in a mixed solvent of water and ethanol was added. The resultant was heated under reflux for 1 h, followed by cooling and then extracting with dichloromethane. The organic layer was washed with pure water until a result of the AgNO₃ test became negative. After drying the organic layer with anhydrous sodium sulfate, the solvent was removed to thereby obtain a colorless liquid of 5.53 g of a colorless liquid of bis(nonafluorobutanesulfonyl)imide-1-dodecyl-2-undecylimidazolium. The yield was 91.1%.

FTIR absorbance peaks of the product are presented below.

Absorbing vibrations were observed at 1,077 cm⁻¹, 1,238 cm⁻¹, 1,355 cm⁻¹, 1,468 cm⁻¹, 1,601 cm⁻¹, 2,859 cm⁻¹, 2,930 cm⁻¹, and 3,268 cm⁻¹.

Peaks of the proton (¹H)NMR and carbon (¹³C)NMR of the obtained compound in CDCl₃ are presented below.

¹H-NMR(CDCl₃, δppm);0.852(t/J=6.8 Hz,3H), 0.858(t/J=6.8 Hz,3H), 1.160-1.400(m,34H), 1.700(quint,t/J=7.7 Hz,2H), 1.770-1.870(m,2H), 2.913(t/J=7.8 Hz,2H), 4.002(t/J=7.6 Hz,2H), 7.085-7.094(m,1H), 7.278-7.291(m,1H)

¹³C-NMR(CDCl₃,δppm);14.051, 22.628, 24.535, 26.336, 27.524, 28.923, 29.019, 29.288, 29.470, 29.498, 29.537, 30.064, 31.856, 47.868, 119.279, 120.601, 147.251

The product was determined as bis(nonafluorobutanesulfonyl)imide-1-dodecyl-2-undecylimidazolium from the spectra above.

Note that, a pKa of an acid (bis(nonafluorobutanesulfonyl)imide) that was a source of the conjugate base in the bis(nonafluorobutanesulfonyl)imide-1-dodecyl-2-undecylimidazolium was 0.0 in acetonitrile.

### Example 3A

### Synthesis of nonafluorobutanesulfonic acid-1-octadecyl-2-undecylimidazolium

A synthesis of nonafluorobutanesulfonic acid-1-octadecyl-2-undecylimidazolium was performed according to the following scheme.

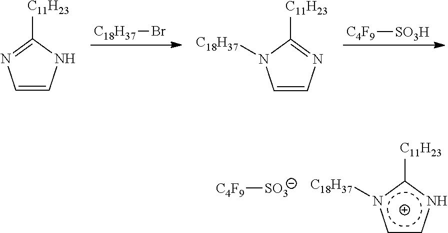

A flask was charged in 200 g of toluene with 15.06 g of 2-undecylimidazole, 22.62 g of octadecyl bromide, 5.00 g of potassium hydroxide, and the mixture was heated under reflux for 12 hours. After returning the temperature to room temperature, the solvent was removed. The resultant was purified by silica gel column chromatography using a mixed solvent of n-hexane and ethyl acetate [n-hexane: ethyl acetate (volume ratio)=9:1] as an eluent, to obtain 29.00 g of 1-octadecyl-2-undecylimidazolium which was a colorless liquid. The yield was 90.2%.

As a result of mass spectrometry using a gas chromatography-mass spectrometer (6890N/5975MSD) available from AGILENT Co., the molecular ion peak appeared at 474 and the gas chromatographic purity was 98.9% or greater.

Peaks of the proton (¹H)NMR and carbon (¹³C)NMR of the obtained compound in deuterated chloroform are presented below.

1H-NMR(CDCl_{3,δppm);}0.846(t/J=7.0 Hz,6H), 1.170-1.390(m,46H), 1.640-1.760(m,4H), 2.599(t/J=7.8 Hz,2H), 3.772(t/J=7.2 Hz, 2H),6.760(d/J=1.6 Hz, 1H),6.890(d/J=1.6 Hz, 1H)

13C-NMR(CDCl₃,δppm):14.080, 22.647, 26.614, 26.806, 28.013, 29.144, 29.326, 29.383, 29.422, 29.498, 29.613, 29.661, 30.945, 31.875, 45.664, 118.608, 126.993, 148.123

The product was determined as 1-octadecyl-2-undecylimidazole from the spectra above.

In ethanol, 4.70 g of 1-octadecyl-2-undecylimidazole was dissolved. To the resultant solution, a solution prepared by dissolving 3.01 g of nonafluorobutanesulfonic acid was added. The resultant was heated under reflux for 1 h. After cooling, the solvent was removed. The residue was extracted with dichloromethane. The organic layer was sufficiently washed with pure water. After drying the organic layer with anhydrous sodium sulfate, the solvent was removed. The resultant was dried in vacuum at 100° C. for 65 hours, to thereby obtain pale yellow crystals of 7.04 g of nonafluorobutanesulfonic acid-1-octadecyl-2-undecylimidazolium. The yield was 91.7%.

FTIR absorbance peaks of the product are presented below.

Absorbing vibrations were observed at 1,060 cm⁻¹, 1,135 cm⁻¹, 1,236 cm⁻¹, 1,277 cm⁻¹, 1,354 cm⁻¹, 1,471 cm⁻¹, 1,520 cm⁻¹, 1,605 cm⁻¹, 2,52 cm⁻¹, 2,919 cm⁻¹ , 3,083 cm⁻¹, 3,148 cm⁻¹, and 3,220 cm⁻¹.

Peaks of the proton (¹H)NMR and carbon (¹³C)NMR of the obtained compound in deuterated chloroform are presented below.

1H-NMR(CDCl₃,δppm);0.849(t/J=6.8 Hz,3H), 0.852(t/J=7.2 Hz,3H), 1.180-1.410(m,46H), 1.670-1.760(m,2H), 1.804(quint/J=6.8 Hz,2H), 2.922(t/J=7.8 Hz,2H), 3.993(t/J=7.4 Hz,2H), 7.081(t/J=2.0 Hz,1H), 7.282(t/J=2.4 Hz,1H)

13C-NMR(CDCl₃,δppm);14.070, 14.099,22.647, 22.666, 24.640, 26.355, 27.285, 28.962, 29.029, 29.278, 29.335, 29.460, 29.498, 29.518, 29.556, 29.671, 30.083, 31.856, 31.894, 47.715, 119.135, 120.554, 147.251

The product was determined as nonafluorobutanesulfonic acid-1-octadecyl-2-undecylimidazolium from the spectra above.

Note that, a pKa of an acid (nonafluorobutanesulfonic acid) that was a source of the conjugate base in the nonafluorobutanesulfonic acid-1-octadecyl-2-undecylimidazolium was 0.7 in acetonitrile.

### Example 4A

### Synthesis of bis(nonafluorobutanesulfonyl)imide-1-octadecyl-2-undecylimidazolium

A synthesis of bis(nonafluorobutanesulfonyl)imide-1-octadecyl-2-undecylimidazolium was performed according to the following scheme.

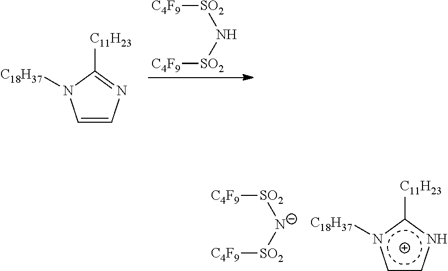

3.01 g of 1-octadecyl-2-undecylimidazole synthesized in Example 3A was dissolved in ethanol, and 3.92 g of potassium bis(nonafluorobutanesulfonyl)imide was dissolved in the ethanol. The resultant was heated under reflux for 1 h. After cooling, the solvent was removed. The residue was extracted with dichloromethane. The organic layer was sufficiently washed with pure water. After drying the organic layer with anhydrous sodium sulfate, the solvent was removed. The resultant was dried in vacuum at 100° C. for 20 hours, to thereby obtain 6.16 g of bis(nonafluorobutanesulfonyl)imide-1-octadecyl-2-undecylimidazolium which was a pale yellow liquid. The yield was 91.9%.

FTIR absorbance peaks of the product are presented below.

Absorbing vibrations were observed at 1,077 cm⁻¹, 1,200 cm⁻¹, 1,217 cm⁻¹ , 1,237 cm⁻¹, 1,354 cm⁻¹, 1,467 cm⁻¹, 1,520 cm⁻¹, 1,601 cm⁻¹, 2,856 cm⁻¹ , 2,927 cm⁻¹ , 3,111 cm⁻¹, 3,170 cm⁻¹, and 3,267 cm⁻¹.

Peaks of the proton (¹H)NMR and carbon (¹³C)NMR of the obtained compound in deuterated chloroform are presented below.

1H-NMR(CDCl₃,δppm);0.851(t/J=6.8 Hz,3H), 0.857(t/J=6.8 Hz, 3H), 1.180-1.400(m,46H), 1.692(quint/J=7.4 Hz,2H), 1.760-1.870(m,2H), 2.904(t/J=7.8 Hz,2H), 4.019(t/J=7.4 Hz,2H), 7.153(t/J=2.2 Hz,1H), 7.246(t/J=2.2 Hz,1H), 11.954(brs,1H)

13C-NMR(CDCl₃,δppm);14.022, 14.070,22.618, 22.666, 24.496, 26.298, 27.448, 28.943, 28.990, 29.259, 29.288, 29.345, 29.470, 29.498, 29.565, 29.680, 30.054, 31.837, 31.904, 47.868, 118.972, 120.937, 147.117

The product was determined as bis(nonafluorobutanesulfonyl/imide-1-octadecyl-2-undecylimidazolium from the spectra above.

Note that, a pKa of an acid [bis(nonafluorobutanesulfonyl)imide] that was a source of the conjugate base in the bis(nonafluorobutanesulfonyl)imide-1-octadecyl-2-undecylimidazolium was 0.0 in acetonitrile.

### Comparative Example 1A

### Synthesis of nonafluorobutanesulfonic acid-1-octadecylimidazolium

Nonafluorobutanesulfonic acid-1-octadecylimidazolium was synthesized according to the following scheme.

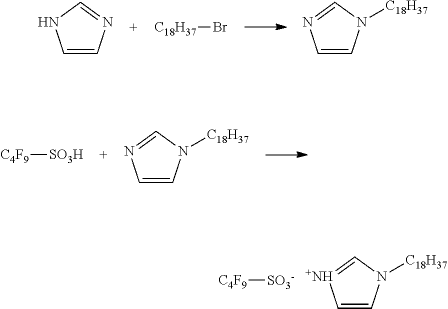

1-Octadecylimidazole was synthesized in the following manner.

Specifically, 3 g of imidazole was dissolved in 100 mL of acetonitrile. 14.9 g of octadecylbromide and 2.51 g of potassium hydroxide were added to the resultant solution. The resultant was reflux for 4 hours under heating with stirring. After removal of the solvent, the resultant was extracted with dichloromethane and purified through column chromatography. As a result of analysis by gas chromatography, the purity was 98.5% or greater.

Subsequently, 3.27 g of the synthesized 1-octadecylimidazole was dissolved in 50 mL of ethanol. To the resultant solution, an ethanol solution of 3.05 g of nonafluorobutanesulfonic acid was gradually added dropwise. After completion of the dropwise addition, the resultant was stirred for 30 minutes and then was heated under reflux for 1 hour. After removal of the solvent, the resultant was recrystallized using a mixed solvent of ethanolln-hexane, to thereby obtain colorless nonafluorobutanesulfonic acid-1-octadecylimidazolium. The yield was 95%.

Assignments of the FTIR spectrum of the product are presented below.

Absorbing vibrations were observed at 1,134 cm⁻¹, 1,355 cm⁻¹ , 1,246 cm⁻¹, 1,470 cm⁻¹ , 2,852 cm⁻¹, 2,920 cm⁻¹, and 3,158 cm⁻¹.

Peaks of the proton (¹H)NMR and carbon (¹³C)NMR of the obtained compound in CDCl₃ are presented below.

1H-NMR(CDCl₃, δppm);0.847(t,3H,J=7.2 Hz), 1.222-1.282(m,30H), 1.790-1.890(m,2H), 4.181(t/J=7.2 Hz,2H), 7.189(dd/J=1.8 Hz,3.8 Hz,1H), 7.444(dd/J=1.8 Hz,3.8 Hz,1H), 8.866(dd/J=1.8 Hz,3.8 Hz,1H), 13.200(brs,1H)

13C-NMR(CDCl₃,δppm);14.055, 22.648, 26.113, 28.875, 29.272, 29.318, 29.440, 30.142, 31.882, 49.847, 122.500, 122.851, 135.015

It was able to be confirmed from the above that nonafluorobutanesulfonic acid-1-octadecylimidazolium was synthesized.

Note that, a pKa of an acid [nonafluorobutanesulfonic acid] that was a source of the conjugate base in the nonafluorobutanesulfonic acid-1-octadecylimidazolium was 0.7 in acetonitrile.

### Comparative Example 2A

### Synthesis of hexafluorocyclopropane-1,3-bis(sulfonyl)imide-1-butyl-3-n-octadecylimidazolium

For comparison, a synthesis of hexafluorocyclopropane-1,3-bis (sulfonyl)imide-1-butyl-3-n-octadecylimidazolium was performed according to the following scheme.

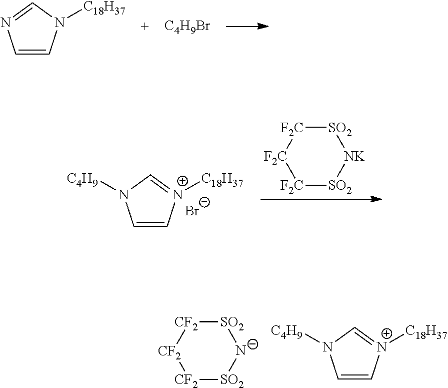

10.7 g of 1-octadecylimidazole synthesized in Comparative Example 1A and 6.03 g of bromobutane were dissolved in acetonitrile, and the resultant solution was heated under reflux for 5 hours. After removal of the solvent, the resultant was recrystallized from a mixed solvent of n-hexane and ethanol, to thereby obtain 1-butyl-3-octadecylimidazolium bromide. 4.57 g of this bromide was dissolved in ethanol. To the resultant solution, an ethanol solution of 3.31 g of potassium hexafluorocyclopropane-1,3-bis(sulfonyl)imide was added. The resultant was stirred to form colorless precipitates. This solution was heated under reflux for 1 hour, and the solvent was removed after cooling. Dichloromethane was added to the resultant and the dissolved matter was filtrated. The organic layer was washed with pure water until a result of the AgNO3 test became negative. After drying, the resultant was recrystallized from a mixed solvent of n-hexane and ethanol, to thereby obtain 6.00 g of hexafluorocyclopropane-1,3-bis(sulfonyl)imide-1-butyl-3-n-octadecylimidazolium, which was colorless crystals. The yield was 90%.

FTIR absorbance peaks of the product are presented below.

Absorbing vibrations were observed at 1,091 cm⁻¹, 1,161 cm⁻¹, 1,356 cm⁻¹, 1,470 cm⁻¹, 1,560 cm⁻¹, 2,850 cm⁻¹, and 2,919 cm⁻¹.

Peaks of the proton (¹H)NMR and carbon (¹³C)NMR of the obtained compound in deuterated chloroform and the assignments are presented below.

1H-NMR(CDCl₃,δppm);0.850(t,3H,J=7.2 Hz), 0.941(t,3H,J=7.2 Hz), 1.170-1.410(m,32H), 1.835(quint,J=7.2 Hz,4M, 4.160(m,4H, 7.267(d,1H,J=2.1 Hz), 7.294(d, 1H,J=2.1 Hz), 8.749(s,1H)

13C-NMR(CDCl₃,δppm);13.254, 14.085, 19.351, 22.663, 26.113, 28.853, 29.303, 29.333, 29.448, 29.570, 29.631, 29.677, 30.127, 31.898, 32.004, 49.977, 50.244, 122.179, 122.263, 135.473

The product was determined as hexafluorocyclopropane-1,3-bis(sulfonyl)imide-1-butyl-3-n-octadecylimidazolium from the spectra above.

Note that, a pKa of an acid [hexafluorocyclopropane-1,3-bis(sulfonyl)imide] that was a source of the conjugate base in the hexafluorocyclopropane-1,3-bis(sulfonyl)imide-1-butyl-3-n-octadecylimidazolium was −0.8 in acetonitrile.

### Comparative Example 3A

### Synthesis of nonafluorobutanesulfonic acid-2-heptadecylimidazole

Nonafluorobutanesulfonic acid-2-heptadecylimidazole was synthesized according to the following scheme.

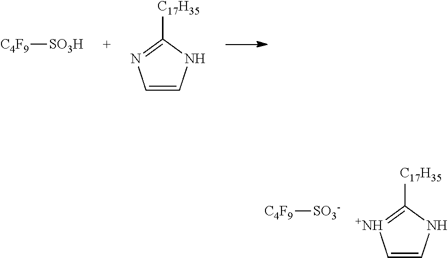

A raw material, 2-heptadecylimidazole, was purchased from SHIKOKU CHEMICALS CORPORATION, and was used after recrystallizing with ethanol. Since thermal stability improved by improving the purity from 93.0% to 98.5% through recrystallization, the purified product through recrystallization was used as the 2-heptadecylimidazole used below as a synthesis raw material. 5.10 g of 2-heptadecylimidazole was dissolved in 100 ml of ethanol. To the resultant solution, 5.00 g of nonafluorobutanesulfonic acid was gradually added dropwise. After completion of the dropwise addition, the resultant was stirred for 30 minutes and then was heated under reflux for 1 hour. After removal of the solvent, the resultant was recrystallized using a mixed solvent of ethanol/n-hexane, to thereby obtain colorless nonafluorobutanesulfonic acid-2-heptadecylimidazole salt. The yield was 95%.

FTIR absorbance peaks of the product are presented below.

Absorbing vibrations were observed at 1,135 cm^{31 1}, 1,356 cm^{31 1}, 1,238 cm^{31 1}, 1,471 cm⁻¹, 2,850 cm⁻¹, 2,918 cm⁻¹, and 3,160 cm⁻¹.

Peaks of the proton (¹H)NMR and carbon (¹³C)NMR of the obtained compound in deuterated chloroform and the assignments are presented below.

1H-NMR(CDCl₃,δppm);0.847(t,3H,J=6.8 Hz), 1.160-1.340(m,28H), 1.710-1.809(m,2H), 2.943(t;J=7.5 Hz,2H), 7.130(s,2H), 11.150(brs,2H)

13C-NMR(CDCl₃,δppm);14.055, 22.663, 25.777, 27.395, 28.875, 28.967, 29.349, 29.379, 29.532, 29.654, 29.684, 31.913, 118.409, 148.584

The product was determined as nonafluorobutanesulfonic acid-2-heptadecylimidazolium from the spectra above.

Note that, a pKa of an acid [nonafluorobutanesulfonic acid] that was a source of the conjugate base in the nonafluorobutanesulfonic acid-2-heptadecylimidazolium was 0.7 in acetonitrile.

### Comparative Example 4A

### Synthesis of nonafluorobutanesulfonic acid-1-octadecyl-2-heptadecyimidazole

A synthesis of nonafluorobutanesulfonic acid-1-octadecyl-2-heptadecyimidazole was performed according to the following scheme.

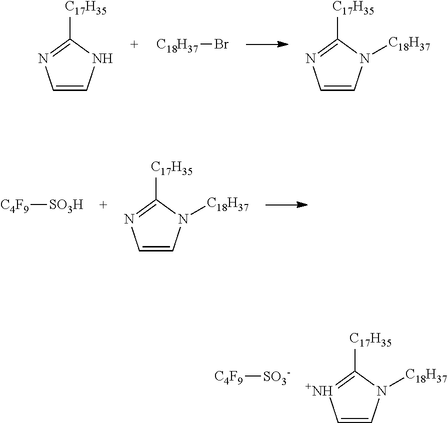

9.18 g of 2-heptadecyimidazole, 9.99 g of octadecyl bromide, and 1.68 g of potassium hydroxide were added to 100 ml of acetonitrile and 100 ml of toluene. The resultant was heated under reflux for 3 hours. The reaction mixture was filtrated to removed the formed salts, and the solvent was removed with an evaporator. The unreacted raw material was separated through silica gel column chromatography using a mixed solvent of n-hexane/ethyl acetate=9/1 (volume ratio), to thereby obtain 14.5 g of a target product,

1-octadecyl-2-heptadecyimidazole at a gas chromatographic purity of 98% or greater.

4.45 g of this imidazole was dissolved in ethanol. To the resultant solution, a solution prepared by dissolving 2.40 g of nonafluorobutanesulfonic acid in ethanol was gradually added dropwise. After completion of the dropwise addition, the resultant was stirred for 30 minutes and then was heated under reflux for 1 hour. is After removal of the solvent, the resultant was recrystallized from a mixed solvent of ethanol/n-hexane, to thereby obtain 6.43 g of colorless nonafluorobutanesulfonic acid-1-octadecyl-2-heptadecylimidazole salt. The yield was 94%.

FTIR absorbance peaks of the product are presented below.

Absorbing vibrations were observed at 1,135 cm⁻¹, 1,279 cm⁻¹ , 1,357 cm⁻¹ , 1,472 cm⁻¹ , 2,851 cm⁻¹ , 2,918 cm⁻¹, and 3,152 cm⁻¹.

Peaks of the proton (¹H)NMR and carbon (¹³C)NMR of the obtained compound in deuterated chloroform and the assignments are presented below.

1H-NMR(CDCl₃,δppm);0.848(t,6H,J=6.8 Hz), 1.171-1.307(m,58H), 1.702-1.817(m,4H), 2.908(t,J=7.5 Hz,2H), 4.003(t,J=7.5 Hz,2H), 7.139(t,J=1.5 Hz,1H), 7.255(t,J=1.5 Hz,1H),13.285(brs,1H)

13C-NMR(CDCl₃, δppm);14.055, 22.648, 24.602, 26.326, 27.196, 28.952, 28.998, 29.333, 29.455, 29.516, 29.562, 29.669, 30.051, 31.882, 47.725, 118.928, 120.866, 147.149

The product was determined as nonafluorobutanesulfonic acid-1-octadecyl-2-heptadecylimidazolium from the spectra above.

Note that, a pKa of an acid [nonafluorobutanesulfonic acid] that was a source of the conjugate base in the nonafluorobutanesulfonic acid-1-octadecyl-2-heptadecylimidazolium was 0.7 in acetonitrile.

### Comparative Example 5A

### Synthesis of pentadecafluorooctanoic acid octadecyl ammonium

For comparison, a synthesis of pentadecafluorooctanoic acid octadecyl ammonium was performed according to the following scheme.

C₇F₁₅—COOH+C₁₈H₃₇—NH₂→C₇F₁₅—COO⁻H₃N⁺—C₁₈H₃₇

In ethanol, 4.14 g of pentadecafluorooctanoic acid and 2.69 g of octadecylamine were added, and the resultant mixture was heated under reflux for 1 h. After removing the solvent from the resultant, the resultant was recrystallized from a mixed solvent of n-hexane and ethanol, to thereby obtain 6.23 g of colorless plate crystals. The yield was 92.0%.

FTIR absorbance peaks of the product are presented below.

Absorbing vibrations were observed at 1,141 cm⁻¹, 1,201 cm⁻¹ , 1,232 cm⁻¹, 1,473 cm⁻¹, 1,677 cm⁻¹, 2,851 cm⁻¹ , 2,918 cm⁻¹, and 3,000⁻¹ to 3,325 cm⁻¹.

Peaks of the proton (¹H)NMR and carbon (¹³C)NMR of the obtained compound in deuterated methanol are presented below.

1H-NMR(CD₃OD,δppm);0.890(t/J=6.6 Hz,3H), 1.214-1.408(m, 30H), 1.590-1.690(m,2H), 2.896(t/J=7.5 Hz,2H), 4.891(brs)

13C-NMR(CD₃OD,δppm);14.444, 23.740, 27.464, 28.578, 30.242, 30.486, 30.516, 30.669, 30.791, 33.081, 40.758

The product was determined as pentadecafluorooctanoic acid octadecyl ammonium from the spectra above.

Note that, a pKa of an acid [pentadecafluorooctanoic acid] that was a base of the conjugate base in the pentadecafluorooctanoic acid octadecyl ammonium was 12.7 in acetonitrile.

The ionic liquids synthesized in Examples and Comparative Examples above are summarized below.

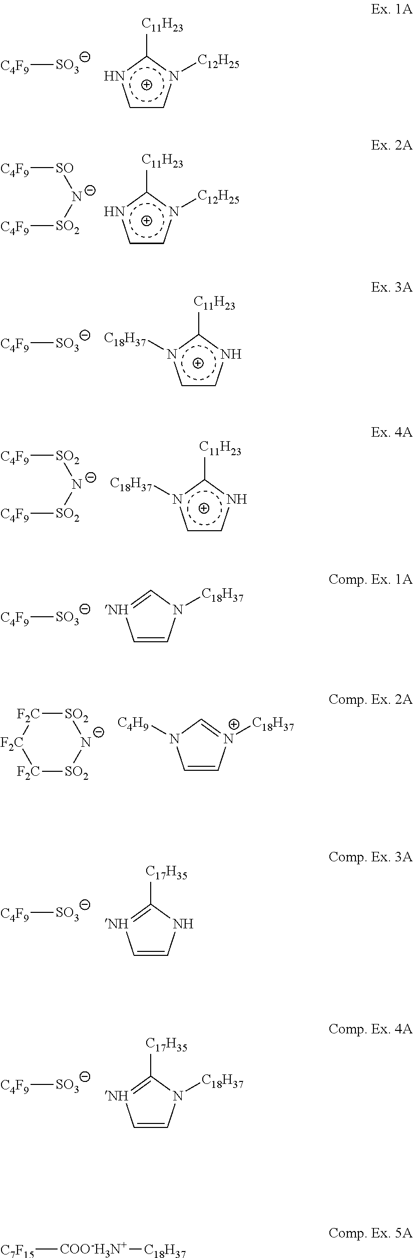

### Example 1B to Example 5B and Comparative Example 1B to Comparative Example 7B

### Solubility Measurement Results to Solvents

Solubility tests were performed on the ionic liquids synthesized in Examples and Comparative Examples, Z-DOL, and Z-TETRAOL, using, as a fluorine-based solvent, Vertrel XF [CF₃(CHF)₂CF₂CF₃] available from Du Pont-Mitsui Fluorochemicals Company, Ltd., and special grade chemical n-hexane available from JUNSEI CHEMICAL CO., LTD.

To a predetermined amount of Vertrel XF or n-hexane, the ionic liquid was added. Ultrasonic waves were applied to the resultant mixture for 5 minutes, followed by leaving the mixture to stand for 1 day. The solubility was visually observed.

Specifically, 0.2 parts by mass of each ionic liquid, Z-DOL, or Z-TETRAOL was added to 100 parts by mass of Vertrel XF (25° C.). After applying ultrasonic waves to the resultant for 5 minutes, the resultant was left to stand for 1 day. Thereafter, the solubility was visually observed and evaluated based on the following evaluation criteria. In the case of n-hexane, similarly, 0.5 parts by mass of each ionic liquid, Z-DOL, or Z-TETRAOL was added to 100 parts by mass of n-hexane at 25° C. Similarly, after applying ultrasonic waves to the resultant for 5 minutes, the resultant was left to stand for 1 day. Thereafter, the solubility was visually observed and evaluated based on the following evaluation criteria.

Note that, the solubility was visually observed, and it was judged as soluble when it was transparent. Moreover, it was judged as not soluble (insoluble) when it was opaque or insoluble components were observed.

The results are presented in Table 2.
[Evaluation Criteria]

### <<Vertrel XF>>

0.2% by mass or greater:

Soluble with addition of 0.2 parts by mass
Less than 0.2% by mass:

Insoluble with addition of 0.2 parts by mass

<<n-Hexane>>
0.5% by mass or greater:

Soluble with addition of 0.5 parts by mass
Less than 0.5% by mass:

Insoluble with addition of 0.5 parts by mass

TABLE 2

Solubility

| Exam- | Synthesized | Fluorine-based | |
|---|---|---|---|
| ples | product | solvent | n-Hexane |
| Ex. 1B | Ex. 1A | 0.2% by mass | 0.5% by mass |
| | | or greater | or greater |
| Ex. 2B | Ex. 2A | 0.2% by mass | 0.5% by mass |
| | | or greater | or greater |
| Ex. 3B | Ex. 3A | 0.2% by mass | 0.5% by mass |
| | | or greater | or greater |
| Ex. 4B | Ex. 4A | 0.2% by mass | 0.5% by mass |
| | | or greater | or greater |
| Comp. | Comp. | less than 0.2% | less than 0.5% |
| Ex. 1B | Ex. 1A | by mass | by mass |
| Comp. | Comp. | less than 0.2% | less than 0.5% |
| Ex. 2B | Ex. 2A | by mass | by mass |
| Comp. | Comp. | less than 0.2% | less than 0.5% |
| Ex. 3B | Ex. 3A | by mass | by mass |
| Comp. | Comp. | less than 0.2% | less than 0.5% |
| Ex. 4B | Ex. 4A | by mass | by mass |
| Comp. | Comp. | less than 0.2% | 0.5% by mass |
| Ex. 5B | Ex. 5A | by mass | or greater |
| Comp. | Z-DOL | 0.2% by mass | less than 0.5% |
| Ex. 6B | | or greater | by mass |
| Comp. | Z-TETRAOL | 0.2% by mass | less than 0.5% |
| Ex. 7B | | or greater | by mass |

The solubility of the ionic liquid of Example 1A was 0.2% by mass or greater to Vertrel XF and 0.5% by mass or greater to n-hexane.

The solubility of the ionic liquid of Example 2A was 0.2% by mass or greater to Vertrel XF and 0.5% by mass or greater to n-hexane.

The solubility of the ionic liquid of Example 3A was 0.2% by mass or greater to Vertrel XF and 0.5% by mass or greater to n-hexane.

The solubility of the ionic liquid of Example 4A was 0.2% by mass or greater to Vertrel XF and 0.5% by mass or greater to n-hexane.

The solubility of the ionic liquids of Comparative Example 1A to Comparative Example 4A was less than 0.2% by mass to Vertrel XF and less than 0.5% by mass to n-hexane. The solubility of the ionic liquid of Comparative Example 5A was less than 0.2% by mass to Vertrel XF but was 0.5% by mass or greater to n-hexane. The solubility of Z-DOL and Z-TETRAOL was 0.2% by mass or greater to Vertrel XF but was less than 0.5% by mass to n-hexane.

As is understood from this, the ionic liquids used in the Examples were improved in solubility to Vertrel XF and n-hexane which are non-polar solvents. Considering the fact that the materials widely used as lubricants are long-chain fatty acid or ester thereof, this means that the ionic liquids can exhibit their effects as an additive. In Examples 1B and 2B using the imidazole derivatives having the hydrocarbon lengths of 11 and 12, the ionic liquids were improved in solubility to Vertrel XF which is a fluorine-based solvent, and thus such lubricants are enough to be used in production process for hard disks. By comparing Examples 3B and 4B with Comparative Example 4B, it is found that solubility was more improved by using a short hydrocarbon chain having 11 or less carbon atoms as one of the two hydrocarbons contained in the imidazolium skeleton which was the conjugate acid. Also, as is understood by comparing Examples 3B and 4B with Comparative Example 1B and Comparative Example 3B, solubility was improved when introducing two hydrocarbon chains whereas solubility was insufficient when introducing only one hydrocarbon chain. Similarly, as is understood by comparing Examples 3B and 4B with Comparative Example 2B, solubility was insufficient when two hydrocarbons were contained but one of the hydrocarbon chains had 4 carbon atoms.

As is understood from Comparative Example 1B to Comparative Example 3B, the imidazole-based ionic liquid having one hydrocarbon, the imidazole-based ionic liquid having two long-chain hydrocarbons introduced, or the imidazole-based ionic liquid having two hydrocarbon chains one of which was a short hydrocarbon chain having 4 carbon atoms was low in solubility to Vertrel XF and n-hexane. That is, it is found that a molecular design effective to solubility to a solvent is introducing two or more hydrocarbons each having 6 or more carbon atoms where one of the hydrocarbons has 14 or less carbon atoms.

From the results the present inventors obtained through consideration, it was found that solubility to fluorine-based solvents and hydrocarbon-based solvents was improved by introducing, into the ionic liquids, two or more long-chain hydrocarbons where one of the hydrocarbons had 14 or less carbon atoms. Also, the ionic liquids having bis(nonafluorobutanesulfonyl)imide as an anion are all is higher in solubility than those having nonafluorobutanesulfonic acid.

### Example 1C

### <Measurement Result of Thermal Stability>

The 5% weight reduction temperature, 10% weight reduction temperature, and 20% weight reduction temperature of nonafluorobutanesulfonic acid-1-dodecyl-2-undecylimidazolium were 349.4° C., 374.1° C., and 398.9° C., respectively. Compared with the commercial products used as comparative examples and known as a lubricant generally used for magnetic recording media, it was found that the weight reduction temperatures were higher by 170° C. or greater than those of perfluoropolyether Z-DOL (Comparative Example GC) and were also 2s higher by 100° C. or greater than those of Z-TETRAOL (Comparative Example 7C).

### Example 2C

### <Measurement Result of Thermal Stability>

The 5% weight reduction temperature, 10% weight reduction temperature, and 20% weight reduction temperature of bis(nonafluorobutanesulfonyl)imide-1-dodecyl-2-undecylimidazolium were 337.7° C., 361.6° C., and 383.8° C., respectively. Compared with the commercial products perfluoropolyether Z-DOL (Comparative Example 6C) and Z-TETRAOL (Comparative Example 7C), it was found that the thermal stability was improved by about 160° C. or greater and 100° C. or greater, respectively.

### Example 3C

### <Measurement Result of Thermal Stability>

The 5% weight reduction temperature, 10% weight reduction temperature, and 20% weight reduction temperature of nonafluorobutanesulfonic acid-1-octadecyl-2-undecylimidazolium were 328.3° C., 368.1° C., and 396.7° C., respectively. Compared with the commercial products used as comparative examples and known as a lubricant generally used for magnetic recording media, it was found that the weight reduction temperatures were higher by 170° C. or greater than those of perfluoropolyether Z-DOL (Comparative Example 6C) and were also higher by 80° C. or greater than those of Z-TETRAOL (Comparative Example 7C).

### Example 4C

### <Measurement Result of Thermal Stability>

The 5% weight reduction temperature, 10% weight reduction temperature, and 20% weight reduction temperature of bis(nonafluorobutanesulfonyl)imide-1-octadecyl-2-undecylimidazolium were 329.1° C., 362.4° C., and 388.4° C., respectively. Compared with the commercial products perfluoropolyether Z-DOL (Comparative Example 6C) and Z-TETRAOL (Comparative Example 7C), it was found that the thermal stability was improved by about 160° C. or greater and 90° C. or greater, respectively.

### Comparative Example 1C

### <Measurement Result of Thermal Stability>

The 5% weight reduction temperature, 10% weight reduction temperature, and 20% weight reduction temperature of nonafluorobutanesulfonic acid-1-octadecylimidazolium were 349.3° C., 375.0° C., and 397.5° C., respectively. Since the nonafluorobutanesulfonic acid-octadecyl ammonium was an ionic liquid, the thermal stability was high compared with the commercial products, perfluoropolyether Z-DOL (Comparative Example GC) and Z-TETRAOL Comparative Example 7C.

### Comparative Example 2C

### <Measurement Result of Thermal Stability>

The 5% weight reduction temperature, 10% weight reduction temperature, and 20% weight reduction temperature of hexafluorocyclopropane-1,3-bis (sulfonyl)imide-1-butyl-3-n-octadecylimidazolium were 347.2° C., 367.0° C., and 387.8° C., respectively. Since the hexafluorocyclopropane-1,3-bis(sulfonyl)imide-1-butyl-3-n-octadecylimidazolium was an ionic liquid, the thermal stability was high compared with the commercial products, perfluoropolyether Z-DOL (Comparative Example 6C) and Z-TETRAOL Comparative Example 7C.

### Comparative Example 3C

### <Measurement Result of Thermal Stability>

The 5% weight reduction temperature, 10% weight reduction temperature, and 20% weight reduction temperature of nonafluorobutanesulfonic acid-2-heptadecylimidazole were 365.4° C., 390.5° C., and 414.3° C., respectively. Since the nonafluorobutanesulfonic acid-2-heptadecylimidazole was an ionic liquid, the thermal stability was high compared with the commercial products, perfluoropolyether Z-DOL (Comparative Example 6C) and Z-TETRAOL (Comparative Example 7C).

### Comparative Example 4C

### <Measurement Result of Thermal Stability>

The 5% weight reduction temperature, 10% weight reduction temperature, and 20% weight reduction temperature of nonafluorobutanesulfonic acid-1-octadecyl-2-heptadecyimidazole were 338.2° C., 365.9° C., and 390.1° C., respectively. Since the nonafluorobutanesulfonic acid-1-octadecyl-2-heptadecyimidazole was an ionic liquid, the thermal stability was high compared with the commercial products, perfluoropolyether Z-DOL (Comparative Example 6C) and Z-TETRAOL (Comparative Example 7C).

### Comparative Example 5C

### <Measurement Result of Thermal Stability>

The 5% weight reduction temperature, 10% weight reduction temperature, and 20% weight reduction temperature of pentadecafluorooctanoic acid octadecyl ammonium were 206.9° C., 215.8° C., and 223.4° C., respectively. Although the pentadecafluorooctanoic acid octadecyl ammonium salt was an ionic liquid, the bonding strength between ions was weak because the pKa of the acid was greater than 10, and hence the pentadecafluorooctanoic acid octadecyl ammonium salt lacked thermal stability. In the case of this Comparative Example, the pentadecafluorooctanoic acid octadecyl ammonium salt was an ionic liquid, but the thermal stability did not significantly improve compared to the commercial products, perfluoropolyether Z-DOL (Comparative Example 6C) and Z-TETRAOL (Comparative Example 7C).

### Comparative Example 6C

### <Measurement Result of Thermal Stability>

A measurement of a commercial product perfluoropolyether Z-DOL having a hydroxyl group at a terminal and a molecular weight of about 2,000 as Comparative Example 6C was performed. As a result, the 5% weight reduction temperature, 10% weight reduction temperature, and 20% weight reduction temperature of Z-DOL were 165.0° C., 197.0° C., and 226.0° C., respectively. The weight reduction was caused by evaporation.

### Comparative Example 7C

### <Measurement Result of Thermal Stability>

Perfluoropolyether (Z-TETRAOL) having a few hydroxyl groups at a terminal and a molecular weight of about 2,000, which was a commercial product and typically used as a lubricant for magnetic recording media was used as a lubricant. The 5% weight reduction temperature, 10% weight reduction temperature, and 20% weight reduction temperature of Z-TETRAOL were 240.0° C., 261.0° C., and 282.0° C., respectively. Similar to Z-DOL, the weight reduction was caused by evaporation.

The results of Examples 1C to 4C and Comparative Examples 1C to 7C are summarized in Table 3 together with their melting points.

TABLE 3

| | Synthesis | | | | |
|---|---|---|---|---|---|
| | Examples or | Weight reduction temperature | | | |
| Exam- | Compound | (° C.) | | | Melting |
| ples | Names | 5% | 10% | 20% | point/° C. |
| Ex. 1C | Ex. 1A | 349.4 | 374.1 | 398.9 | 43.3 |
| Ex. 2C | Ex. 2A | 337.7 | 361.6 | 383.8 | <25.0 |
| Ex. 3C | Ex. 3A | 328.3 | 368.1 | 396.7 | 52.6 |
| Ex. 4C | Ex. 4A | 329.1 | 362.4 | 388.4 | <25.0 |
| Comp. | Comp. | 349.3 | 375.0 | 397.5 | 86.2 |
| Ex. 1C | Ex. 1A | | | | |
| Comp. | Comp. | 347.2 | 367.0 | 387.8 | 55.8 |
| Ex. 2C | Ex. 2A | | | | |
| Comp. | Comp. | 365.4 | 390.5 | 414.3 | 60.6 |
| Ex. 3C | Ex. 3A | | | | |
| Comp. | Comp. | 338.2 | 365.9 | 390.1 | 72.3 |
| Ex. 4C | Ex. 4A | | | | |
| Comp. | Comp. | 206.9 | 215.8 | 223.4 | 61.7 |
| Ex. 5C | Ex. 5A | | | | |
| Comp. | Z-DOL | 165.0 | 197.0 | 226.0 | <25.0 |
| Ex. 6C | | | | | |
| Comp. | Z-TETRAOL | 240.0 | 261.0 | 282.0 | <25.0 |
| Ex. 7C | | | | | |

As is clear from the descriptions above, the lubricants based on the imidazole-based ionic liquids having long-chain hydrocarbon groups in Example 1A to Example 4A and Comparative Example 1A to Comparative Example 4A are much superior in thermal stability to Z-DOL and Z-TETRAOL which are commercial perfluoropolyether. As described above, the ionic liquid of Comparative Example 5A used carboxylic acid having a high pKa, and thus the binding force between the ions was weak to give the result of lacking in thermal stability.

Comparison in terms of thermal stability among the imidazole-based ionic liquids having long-chain hydrocarbon groups does not give any systematic difference, but they are believed to have sufficient thermal stability.

The ionic liquids of the Examples are lower in melting point than the ionic liquids of Comparative Examples 1A to 4A, having an advantage that they will find a wider application field as a lubricant.

### Example 1D to Example 4D and Comparative Example 1D to Comparative Example 4D

<Disk Durability test>

Magnetic disks were produced by applying lubricants including the ionic liquids of Examples 1D to 4D and Comparative Examples 1D to 4D, respectively. As presented in Table 4, the CSS measurements of the magnetic disks were greater than 50,000 times, and the CSS measurements after the heating test were also greater than 50,000 times, and the magnetic disks exhibited excellent durability.

### Comparative Example 5D

### <Disk Durability Test>

The magnetic disk was produced using a lubricant including pentadecafluorooctanoic acid octadecyl ammonium salt. As presented in Table 4, the CSS measurement of the magnetic disk was greater than 50,000 times but the CSS measurement of the heating test was 891 times and the durability of the magnetic disk was deteriorated by the heating test. As presented with Comparative Example 12, the pentadecafluorooctanoic acid octadecyl ammonium salt was an ionic liquid, but the pKa of the acid was greater than 10. Therefore, the bonding strength between ions was weak and the thermal stability was deteriorated, and therefore it was considered that the properties after the heating test were deteriorated.

### Comparative Example 6D

### <Disk Durability Test>

The magnetic disk was produced using a lubricant including Z-DOL. As presented in Table 4, the CSS measurement of the magnetic disk was greater than 50,000 times but the CSS measurement of the heating test was the CSS measurement of the heating test was 12,000 times and the durability of the magnetic disk was deteriorated by the heating test.

### Comparative Example 7D

### <Disk Durability Test>

The magnetic disk was produced using a lubricant including Z-TETRAOL. As presented in Table 4, the CSS measurement of the magnetic disk was greater than 50,000 times but the CSS measurement of the heating test was the CSS measurement of the heating test was 36,000 times and the durability of the magnetic disk was deteriorated by the heating test.

The results of Examples 1D to 4D, and Comparative Examples 1D to 7D are summarized in Table 4.

TABLE 4

| | Synthesis | | | | |
|---|---|---|---|---|---|
| | Examples or | | | | |
| Exam- | Compound | | | CSS durability | |
| ples | Names | CSS durability | | after heating | |
| Ex. 1D | Ex. 1A | 25° C., | >50,000 | 25° C., | >50,000 |
| | | 60% RH | | 60% RH | |
| Ex. 2D | Ex. 2A | 25° C., | >50,000 | 25° C., | >50,000 |
| | | 60% RH | | 60% RH | |
| Ex. 3D | Ex. 3A | 25° C., | >50,000 | 25° C., | >50,000 |
| | | 60% RH | | 60% RH | |
| Ex. 4D | Ex. 4A | 25° C., | >50,000 | 25° C., | >50,000 |
| | | 60% RH | | 60% RH | |
| Comp. | Comp. | 25° C., | >50,000 | 25° C., | >50,000 |
| Ex. 1D | Ex. 1A | 60% RH | | 60% RH | |
| Comp. | Comp. | 25° C., | >50,000 | 25° C., | >50,000 |
| Ex. 2D | Ex. 2A | 60% RH | | 60% RH | |
| Comp. | Comp. | 25° C., | >50,000 | 25° C., | >50,000 |
| Ex. 3D | Ex. 3A | 60% RH | | 60% RH | |
| Comp. | Comp. | 25° C., | >50,000 | 25° C., | >50,000 |
| Ex. 4D | Ex. 4A | 60% RH | | 60% RH | |
| Comp. | Comp. | 25° C., | >50,000 | 25° C., | 891 |
| Ex. 5D | Ex. 5A | 60% RH | | 60% RH | |
| Comp. | Z-DOL | 25° C., | >50,000 | 25° C., | 12,000 |
| Ex. 6D | | 60% RH | | 60% RH | |
| Comp. | Z-TETRAOL | 25° C., | >50,000 | 25° C., | 36,000 |
| Ex. 7D | | 60% RH | | 60% RH | |

### Example 1E to Example 4E and Comparative Example 1E to Comparative Example 7E

After producing magnetic tapes using lubricants including the ionic liquids of Examples 1A to 4A, the ionic liquids of Comparative Examples 1A to 5A, Z-DOL, and Z-Tetraol, respectively, the following measurements were performed.
Coefficient of friction of magnetic tape after shuttle run of 100 times:

In the environment having a temperature of −5° C., or in the environment having a temperature of 40° C. and relative humidity of 90%.
Still durability test:

In the environment having a temperature of −5° C., or in the environment having a temperature of 40° C. and relative humidity of 30%.
Shuttle durability test:

In the environment having a temperature of −5° C., or in the environment having a temperature of 40° C. and relative humidity of 90%.
Coefficient of friction of magnetic tape after shuttle run of 100 times after heating test:

In the environment having a temperature of −5° C., or in the environment having a temperature of 40° C. and relative humidity of 90%.
Still durability test after heating test:

In the environment having a temperature of −5° C., or in the environment having a temperature of 40° C. and relative humidity of 30%.
Shuttle durability test after heating test:

In the environment having a temperature of −5° C., or in the environment having a temperature of 40° C. and relative humidity of 90%.

The results of Examples 1E to 4E and Comparative Examples 1E to 7E are summarized in Tables 5-1 and 5-2.

TABLE 5-1

| | | | | | | | | Coefficient of | | | | | |
|---|---|---|---|---|---|---|---|---|---|---|---|---|---|
| | | Coefficient of | | | | | | friction after | | Still durability | | Shuttle | |
| | | friction after | | Still | | Shuttle | | 100 runs after | | after heating | | durability after | |
| | Compound | 100 runs | | durability/min | | durability/times | | heating test | | test/min | | heating/times | |
| Ex. 1E | Ex. 1A | −5° C. | 0.23 | −5° C. | >60 | −5° C. | >200 | −5° C. | 0.24 | −5° C. | >60 | −5° C. | >200 |
| | | 40° C., | 0.24 | 40° C., | >60 | 40° C., | >200 | 40° C., | 0.25 | 40° C., | >60 | 40° C., | >200 |
| | | 90% RH | | 30% RH | | 90% RH | | 90% RH | | 30% RH | | 90% RH | |
| Ex. 2E | Ex. 2A | −5° C. | 0.22 | −5° C. | >60 | −5° C. | >200 | −5° C. | 0.23 | −5° C. | >60 | −5° C. | >200 |
| | | 40° C., | 0.25 | 40° C., | >60 | 40° C., | >200 | 40° C., | 0.26 | 40° C., | >60 | 40° C., | >200 |
| | | 90% RH | | 30% RH | | 90% RH | | 90% RH | | 30% RH | | 90% RH | |
| Ex. 3E | Ex. 3A | −5° C. | 0.20 | −5° C. | >60 | −5° C. | >200 | −5° C. | 0.21 | −5° C. | >60 | −5° C. | >200 |
| | | 40° C., | 0.21 | 40° C., | >60 | 40° C., | >200 | 40° C., | 0.22 | 40° C., | >60 | 40° C., | >200 |
| | | 90% RH | | 30% RH | | 90% RH | | 90% RH | | 30% RH | | 90% RH | |
| Ex. 4E | Ex. 4A | −5° C. | 0.21 | −5° C. | >60 | −5° C. | >200 | −5° C. | 0.22 | −5° C. | >60 | −5° C. | >200 |
| | | 40° C., | 0.22 | 40° C., | >60 | 40° C., | >200 | 40° C., | 0.23 | 40° C., | >60 | 40° C., | >200 |
| | | 90% RH | | 30% RH | | 90% RH | | 90% RH | | 30% RH | | 90% RH | |

TABLE 5-2

| | | | | | | | | Coefficient of | | | | | |
|---|---|---|---|---|---|---|---|---|---|---|---|---|---|
| | | Coefficient of | | | | | | friction after | | Still durability | | Shuttle | |
| | | friction after | | Still | | Shuttle | | 100 runs after | | after heating | | durability after | |
| | Compound | 100 runs | | durability/min | | durability/times | | heating test | | test/min | | heating/times | |
| Comp. | Comp. | −5° C. | 0.20 | −5° C. | >60 | −5° C. | >200 | −5° C. | 0.20 | −5° C. | >60 | −5° C. | >200 |
| Ex. 1E | Ex. 1A | 40° C., | 0.22 | 40° C., | >60 | 40° C., | >200 | 40° C., | 0.23 | 40° C., | >60 | 40° C., | >200 |
| | | 90% RH | | 30% RH | | 90% RH | | 90% RH | | 30% RH | | 90% RH | |
| Comp. | Comp. | −5° C. | 0.23 | −5° C. | >60 | −5° C. | >200 | −5° C. | 0.25 | −5° C. | >60 | −5° C. | >200 |
| Ex. 2E | Ex. 2A | 40° C., | 0.25 | 40° C., | >60 | 40° C, | >200 | 40° C., | 0.27 | 40° C., | >60 | 40° C., | >200 |
| | | 90% RH | | 30% RH | | 90% RH | | 90% RH | | 30% RH | | 90% RH | |
| Comp. | Comp. | −5° C. | 0.18 | −5° C. | >60 | −5° C. | >200 | −5° C. | 0.19 | −5° C. | >60 | −5° C. | >200 |
| Ex. 3E | Ex. 3A | 40° C., | 0.20 | 40° C., | >60 | 40° C., | >200 | 40° C., | 0.21 | 40° C., | >60 | 40° C., | >200 |
| | | 90% RH | | 30% RH | | 90% RH | | 90% RH | | 30% RH | | 90% RH | |
| Comp. | Comp. | −5° C. | 0.20 | −5° C. | >60 | −5° C. | >200 | −5° C. | 0.21 | −5° C. | >60 | −5° C. | >200 |
| Ex. 4E | Ex. 4A | 40° C., | 0.22 | 40° C., | >60 | 40° C., | >200 | 40° C., | 0.23 | 40° C., | >60 | 40° C., | >200 |
| | | 90% RH | | 30% RH | | 90% RH | | 90% RH | | 30% RH | | 90% RH | |
| Comp. | Comp. | −5° C. | 0.21 | −5° C. | >60 | −5° C. | >200 | −5° C. | 0.45 | −5° C. | 12 | −5° C. | 30 |
| Ex. 5E | Ex. 5A | 40° C., | 0.25 | 40° C., | >60 | 40° C., | >200 | 40° C., | 0.51 | 40° C., | 16 | 40° C., | 23 |
| | | 90% RH | | 30% RH | | 90% RH | | 90% RH | | 30% RH | | 90% RH | |
| Comp. | Z-DOL | −5° C. | 0.25 | −5° C. | 12 | −5° C. | 59 | −5° C. | 0.32 | −5° C. | 12 | −5° C. | 46 |
| Ex. 6E | | 40° C., | 0.30 | 40° C., | 48 | 40° C., | 124 | 40° C., | 0.35 | 40° C., | 15 | 40° C., | 58 |
| | | 90% RH | | 30% RH | | 90% RH | | 90% RH | | 30% RH | | 90% RH | |
| Comp. | Z- | −5° C. | 0.22 | −5° C. | 25 | −5° C. | 65 | −5° C. | 0.28 | −5° C. | 23 | −5° C. | 55 |
| Ex. 7E | TETRAOL | 40° C., | 0.26 | 40° C., | 35 | 40° C., | 156 | 40° C., | 0.32 | 40° C., | 31 | 40° C., | 126 |
| | | 90% RH | | 30% RH | | 90% RH | | 90% RH | | 30% RH | | 90% RH | |

In the tables, “>60” of the still durability denotes greater than 60 minutes.

In the tables, “>200” of the shuttle durability denotes greater than 200 times.

The following facts were confirmed.

It was found that the magnetic tapes to which the lubricants including the ionic liquids of Examples 1A to 4A were applied, respectively, had excellent friction resistance, still durability, and shuttle durability.

It was found that the magnetic tapes to which the lubricants including the ionic liquids of Comparative Examples 1A to 4A were applied, respectively, had excellent friction resistance, still durability, and shuttle durability. Since the lubricants in Comparative Examples 1A to 4A were the ionic liquids, the magnetic tapes had excellent durability even after the heating test.

The magnetic tape to which the lubricant including the ionic liquid of Comparative Example 5A was applied indicated excellent friction resistance, still durability, and shuttle durability, but caused significant deterioration of the magnetic tape after the heating test.

It was found that the magnetic tape to which Z-DOL was applied had significant deterioration in still durability and shuttle durability.

It was found that the magnetic tape to which Z-Tetraol was applied had significant deterioration in still durability and shuttle durability.

It was found from Tables 5-1 and 5-2 that excellent heat resistance, and durability of the magnetic tape and magnetic disk could be obtained when the lubricant included the ionic liquid including a conjugate base and a conjugate acid, where the conjugate acid had two or more straight-chain hydrocarbon groups each having 6 or greater carbon atoms, the number of carbon atoms in one of the groups is 14 or less, and a pKa of an acid that was a source of the conjugate base was 10 or less in acetonitrile. Not only achieving excellent thermal resistance and durability of a magnetic recording medium, such lubricants dissolved to n-hexane as a diluent as well as the ionic liquid, which meant that the lubricants could exhibit an effect as an additive to long-chain fatty acid or ester of long-chain fatty acid, which were widely used as lubricants. Some of the lubricants were soluble to Vertrel that was a fluorine-based solvent, and thus use of such lubricants on production process was not problem, particularly when the lubricants were used for hard disks and micromachines.

As is clear from the descriptions above, an ion liquid-based lubricant, which includes an ionic liquid including a conjugate base and a conjugate acid, where the conjugate acid had two or more straight-chain hydrocarbon groups each having 6 or greater carbon atoms, the number of carbon atoms in one of the groups is 14 or less, and a pKa of an acid that was a source of the conjugate base was 10 or less in acetonitrile, has a high decomposition temperature, and high 5%, 10%, and 20% weight reduction temperatures, and has excellent thermal stability. Moreover, the lubricant can maintain excellent lubricity under high temperature conditions compared to conventional perfluoropolyether, and can maintain lubricity over a long period. Accordingly, a magnetic recording medium using the lubricant including the ionic liquid can obtain excellent running performances, abrasion resistance, and durability.

This application claims priority to Japanese application No. 2016-133680, filed on Jul. 5, 2016 and incorporated herein by reference.

What is claimed is:

1. A lubricant comprising:
an ionic liquid including a conjugate base and a conjugate acid,
wherein the conjugate acid includes a straight-chain hydrocarbon group having 6 or greater carbon atoms and a straight-chain hydrocarbon group having 6 or greater but 14 or less carbon atoms, and
wherein a pKa of an acid that is a source of the conjugate base in acetonitrile is 10 or less.

2. The lubricant according to claim 1,
wherein the conjugate acid is represented by General Formula (A) below, 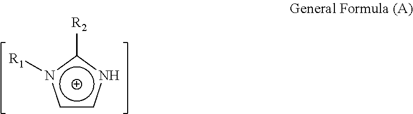
where, in General Formula (A),
R₁ is a group including a straight-chain hydrocarbon group having 6 or greater carbon atoms and R₂ is a group including a straight-chain hydrocarbon group is having 6 or greater but 14 or less carbon atoms: or
R₁ is a group including a straight-chain hydrocarbon group having 6 or greater but 14 or less carbon atoms and R₂ is a group including a straight-chain hydrocarbon group having 6 or greater carbon atoms.

3. The lubricant according to claim 1,
wherein the conjugate base is represented by General Formula (X) or General Formula (Y) below, 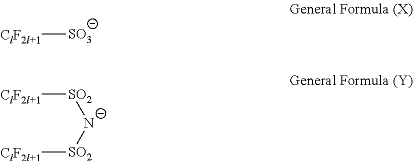
where, in General Formula (X), 1 is an integer of 1 or greater but 12 or less, and
where, in General Formula (Y), 1 is an integer of 1 or greater but 12 or less.

4. The lubricant according to claim 2,
wherein the conjugate base is represented by General Formula (X) or General Formula (Y) below, 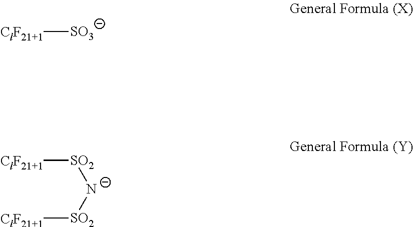
where, in General Formula (X), 1 is an integer of 1 or greater but 12 or less, and
where, in General Formula (Y), 1 is an integer of 1 or greater but 12 or less.

5. A magnetic recording medium comprising:
a non-magnetic support;
a magnetic layer disposed on the non-magnetic support; and
the lubricant according to claim 1, disposed on the magnetic layer.

6. An ionic liquid comprising:
a conjugate base; and
a conjugate acid,
wherein the conjugate acid includes a straight-chain hydrocarbon group having 6 or greater carbon atoms and a straight-chain hydrocarbon group having 6 or greater but 14 or less carbon atoms, and
wherein a pKa of an acid that is a source of the conjugate base in acetonitrile is 10 or less.

7. The ionic liquid according to claim 6,
wherein the conjugate acid is represented by General Formula (A) below, 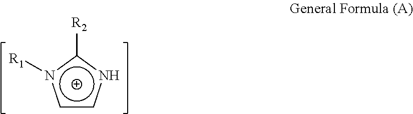
where, in General Formula (A),
R₁ is a group including a straight-chain hydrocarbon group having 6 or greater carbon atoms and R₂ is a group including a straight-chain hydrocarbon group having 6 or greater but 14 or less carbon atoms; or
R₁ is a group including a straight-chain hydrocarbon group having 6 or greater but 14 or less carbon atoms and R₂ is a group including a straight-chain hydrocarbon group having 6 or greater carbon atoms.

8. The ionic liquid according to claim 6,
wherein the conjugate base is represented by General Formula (X) or General Formula (Y) below, 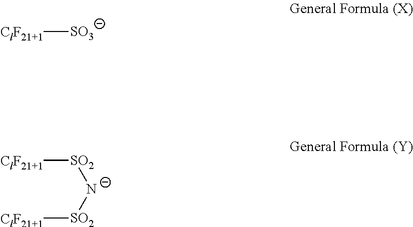
where, in General Formula (X), 1 is an integer of 1 or greater but 12 or less, and
where, in General Formula (Y), 1 is an integer of 1 or greater but 12 or less.

9. The ionic liquid according to claim 7,
wherein the conjugate base is represented by General Formula (X) or General Formula (Y) below, 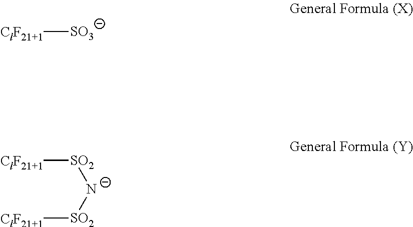
where, in General Formula (X), 1 is an integer of 1 or greater but 12 or less, and
where, in General Formula (Y), 1 is an integer of I or greater but 12 or less.
